(19) United States
(12) Patent Application Publication
(10) Pub. No.: US 2006/0127437 A1
(43) Pub. Date: Jun. 15, 2006

(54) Semisolid system and combination semisolid, multiparticulate system for sealing tissues and/or controlling biological fluids

(75) Inventors: John P. Kennedy, Sarasota, FL (US); Curtis E. Jones II, Savannah, GA (US)

Correspondence Address: BROMBERG & SUNSTEIN LLP, 125 SUMMER STREET, BOSTON, MA, 02110-1618, US

(73) Assignee: MISTY ANDERSON KENNEDY

(21) Appl. No.: 11/009,623

(22) Filed: Dec. 13, 2004

(51) Int. Cl. A61F 13/00

(52) U.S. Cl. 424/422

(57) ABSTRACT

A semisolid system and combination semisolid, multiparticulate system for controlling biological fluids is provided which includes a body fluid control composition. The body fluid control composition is adapted for utilization both as an independent mixture for direct topical application to body tissue for human or veterinary application and for incorporation with a standard medical products in order to facilitate absorption, moisture balance, hemostasis, infection control, and wound healing.

### RELATED APPLICATIONS

The present invention was first described in Disclosure Document Registration No. 539,269 filed on Sep. 29, 2003 under 35 U.S.C. §122, 37 C.F.R. §1.14, and MPEP §1706. There are no previously filed, nor currently any co-pending applications, anywhere in the world.

### BACKGROUND OF THE INVENTION

1. Field of the Invention

The present invention relates generally to fluid absorbent articles and, more particularly, to a semisolid, biological fluid control system.

Accordingly, the development of a semisolid biocompatible system for the control of biological fluid offers a novel alternative to the prior art in the field.

2. Description of the Related Art

The need to control the loss of fluids and maintain a moisture balance for viable biological functions is timeless goal of living organisms. This need may manifest itself out of a simple desire to maintain personal cleanliness or as a medical necessity in the control of hemorrhagic episodes resulting from trauma, disease or medical procedures. In fact the need and use of such products in daily life is widespread. Medical articles or products such as sponges, dressings and bandages absorb bodily fluid exudates and assist in the establishment of hemostasis largely through the physical action of slowing blood flow to promote spontaneous hemostasis. However, the utility of such products in extensive or diffuse injury of highly vascular tissues is largely limited. Newer technologies in wound healing not only absorb exudate, but also manage a moisture balance that promotes faster healing. The application of products that may act at the site of hemorrhage or multiple micro-hemorrhages would offer a significant improvement. The application of such products in the practice of medicine significantly reduce the morbidity and mortality associated with traumatic injury and secondary to both major and minor surgical procedures by reducing the stress associated with incidences of blood loss and primary hypoperfusion of vital organs such as the heart, liver, brain and kidneys. This in turn reduces the need for volume expansion by the use of intravenous fluids and/or blood transfusions. The reduction in the amount of time required to establish, and maintain, hemostasis is shown in the literature to significantly improve the ultimate long-term outcome and prognosis in many surgical procedures and traumatic injuries. Maintenance of a moisture balance at levels optimal for wound healing and sub-optimal for bacterial growth is shown in the literature to significantly improve the healing rates of wounds and minimize opportunities for disease and toxicity caused by bacterial proliferation.

Therefore a product that exhibits the ability to absorb and/or efficiently stop the flow of blood in a wide range of situations would be of great medical significance and commercial interest. Furthermore, a product that also exhibits the capacity to balance moisture levels, a principle goal in modern wound care, would be synergistic and likewise of great medical and commercial interests. Such a product should be easy to administer and exhibit a great deal of flexibility in utilization. In addition such a product should be biocompatible, ideally biodegradable and safe in residence in the human body for extended periods of time if necessary. The present invention teaches the use of such a product that may be produced, in its preferred embodiments, to exist in physical states ranging from a flowable liquid to a highly viscous gel system capable of impeding the flow of blood and/or regulating moisture balances in a wide range of situations. The prior art related to this area teaches the use of products with some like qualities. These are as follows:

U.S. Pat. No. 6,312,725 describes the use of two-part polymer compositions that chemically form covalent linkages when mixed resulting in the formation of adherent gel systems useful as tissue sealants and tissue augmentation, hemostatic agents and for drug delivery systems.

U.S. Pat. Nos. 5,651,982; 5,631,019; 5,607,694 describes multiple component biocompatible sealant compositions, that when mixed in situ, form a fibrin glue with bioadhesive and hemostatic properties containing accompanying liposomal structures for the delivery of active agents.

U.S. Pat. No. 6,568,398 teaches the use of hemostatic compositions comprising microcapsules of granular astringent hemostatic agent microencapsulated with a biocompatible polymer for oral, dermatological or other hemostasis applications.

U.S. Pat. No. 6,162,241 describes the use of a biodegradable, biocompatible synthetic polymer possessing no inherent hemostatic properties with the inclusion of a hemostatic material used as a tissue sealant system.

U.S. Pat. No. 6,537,569 teaches the use of a radiation-crosslinked, biodegradable, synthetic hydrogels that are implanted on or in the body used for hemostasis, tissue augmentation, or closure of vascular punctures or wounds.

U.S. Pat. No. 6,488,952 teaches the use of a semisolid system and combination semisolid, multiparticulate system that formed a liquid crystalline system and demonstrated hemostatic properties.

The prior art in the field generally teaches the use of isolated products in the normal clotting cascade or products that rely on a chemical reaction in preparation or in situ to establish hemostasis. The greatest difference in reference to the present invention is the use of a product with great flexibility that utilizes a lipophilic system consisting of GRAS substances that exert hemostatic properties resulting from physical changes initiated in the system upon contact with blood or and aqueous body fluid. The inventors of U.S. Pat. No. 6,488,952 and the present invention are the same. U.S. Pat. No. 6,488,952 makes broad claims on the system's ability to facilitate hemostasis. The present invention clarifies the utilities for hemostasis including synergistic combinations.

The treatment of superficial wounds also has many of the same general requirements as systems used to promote hemostasis. For example the establishment of local hemostasis is of great importance. In addition, it is highly beneficial that a wound maintains a proper moisture balance while removing excess exudate. This provides an optimum wound healing environment, reduces pain, and provides an environment conducive to autolytic debridement and re-epithelialization. Excessive fluid exudate may promote skin decomposition or maceration and serve as a focus for microbial infection. However a system which promotes the proper hydration status of the superficial tissues and which serves to augment the normal barrier function of the skin would greatly serve to promote rapid, complete healing and reduce the likelihood of secondary bacterial infection. Conventional dressings often fail to properly regulate moisture transmission by removing excess exudate while maintaining a well hydrated environment. An improvement in this area is realized with the advent of hydrogel bandages and systems. These systems place a gel system that is primarily aqueous in nature in close proximity to the wound. Many of these systems are incorporated into conventional spun or woven bandage systems and have the capability to absorb limited amounts of exudates. Prior art in this area include the following:

U.S. Pat. No. 5,858,392 teaches the use of a polyionic hydrogel that is prepared through a chemical reaction of an anionic polysaccharide and a solution of cationic polysaccharide within a support material that is further dried by a process such as lyophilization.

U.S. Pat. No. 5,160,328 teaches the use of a self-adhesive bandage system comprised of a plurality of layers including a backing layer, an adhesive layer and a polyurethane hydrogel layer that is disposed over the second side of the backing layer that is suitable for absorbing bodily fluids.

U.S. Pat. No. 5,620,702 teaches the use of an adhesive bandage, dressing, or suture-like mechanism made from a laminate of flexible rubber and a hydrophilic hydrogel polymer comprised of a cellulosic, polyurethane or polyacrylate base, bonded to one side of the flexible rubber.

U.S. Pat. No. 5,393,798 teaches the use of hydrogel materials made from modified, cross-linked copolymers of poly(methyl vinyl ether/maleic anhydride) suitable for use in bandages utilizing an acceptable support mechanism.

U.S. Pat. No. 6,488,952 teaches the use of a semisolid system and a combination semisolid, multiparticulate system that forms a liquid crystalline system and provides an inherent antimicrobial effect for the treatment of wounds.

And like traditional fluid absorption devices such as conventional secondary sponges, dressings and bandages, hydrogel systems generally fail to address the importance or need for hemostatic agent(s) with effective bodily fluid absorption articles. The present invention differs significantly from the prior art in that the system serves to regulate and maintain a fluid balance very similar to that of normal skin (25%-40% water content). In addition the present invention is comprised primarily of lipophilic material with a final relative composition far more similar to that of normal skin than the highly hydrophilic products currently available. The inventors of U.S. Pat. No. 6,488,952 and the present invention are the same. U.S. Pat. No. 6,488,952 makes broad claims on the system's ability to: (1) treat skin injuries and diseases and (2) resist microbial growth. The present invention clarifies the utilities: (1) for moist wound healing including synergistic combinations and (2) for an anti-infective agent including synergistic combinations.

In addition to common products such as surgical coverings, sponges, dressings and bandages, other absorbent devices are generally known and utilized in various articles of commerce. Well known articles, which employ absorbent structures or components, include disposable diapers, adult incontinence pads and brief, bed pads, and catamenial products such as sanitary napkins, menstrual pads, tampons, and panty liners. Such articles are also the subjects of substantial commercial interest.

Depending upon the application of such products, they will possess diverse constructions, while nevertheless utilizing common components providing comparatively similar functions. The purpose of these articles is to absorb fluids, more specifically bodily fluids, and retain such fluids within the article in order that they may be sanitarily disposed of after use. Thinness is a highly desirable characteristic of these articles, particularly catamenial products, as thinner products are less corpulent, more easily adapted to fit under clothing, and less conspicuous, thus facilitating user comfort when worn, as well as imparting reduced distribution costs on the manufacturer and distributor, requiring less packaging material, and occupying less shelf space.

In order to provide thinner absorbent articles, the prior art teaches the use of fluid-absorbent resins referred to as super absorbent polymers, or SAPs. SAPs are generally lightly crosslinked hydrophilic polymers, which are capable of absorbing and retaining large quantities of discharged body fluids, especially when combined with a fibrous matrix. Such fluid-absorbent resins are commonly available in different chemical forms, including substituted and unsubstituted natural and synthetic polymers, such as hydrolysis products of carboxymethylcellulose, crosslinked polyacrylates, polyvinyl alcohols, sulfonated polystyrenes, polyethylene oxides, polyacrylonitriles, and polyvinylpyrrolidones that generally exhibit absorbent properties through a method consisting of swelling to retain excess fluid. However, the absorption capacity of these SAPs is drastically reduced in electrolytic solutions, such as urine, saline, and blood. The prior art related to the area include the following:

U.S. Pat. No. 5,478,355 teaches the use of an absorbent device having an absorbent core to retain bodily fluids, a cover sheet of porous or netlike construction on a body facing side and an intermediate layer between the cover sheet and the core.

U.S. Pat. No. 5,300,358 teaches the use of biodegradable absorbent structures for sanitary applications in controlling the flow of body fluids comprising an absorbent degradable fibrous core with a back sheet of material that is cold-water soluble but water impermeable.

U.S. Pat. No. 4,232,674 teaches the use of a liquid absorbent device such as a diaper, sanitary towel or tampon includes one or more layers of material exhibiting a capillary action to convey liquid to an absorbent starch based gel.

U.S. Pat. No. 6,488,952 teaches the use of a semisolid system and combination semisolid, multiparticulate system based on a lipid that formed a liquid crystalline system and demonstrated ability to control exudate flow.

Thus, with particularly with respect to catamenial products, the absorbent polymers employed in prior absorbent devices have been inefficient in blood absorption rate, thereby preventing such devices from effectively absorbing bodily fluid discharge, especially in effusive or gushing instances. Further, prior absorbent devices have neglected to address the combination of effective bodily fluid absorption and hemostasis, but rather have focused specifically on fluid absorption. Still further, the association of toxic shock syndrome with the use of tampons is a viable concern. Toxic shock syndrome (TSS) is a severe and sometimes fatal multi-system disease associated with infection or colonization by Staphylococcus aureus bacteria. TSS is believed to be caused by toxic shock syndrome toxin-1, the toxin produced by Staphlococcal strains. Hyperabsorbent tampons may facilitate the infection because their prolonged intra-vaginal use alters the fluid-hydration status of the vaginal mucosa creating an environment that enhances bacterial growth. Also, the super absorbent fibers in some tampons may cause microscopic tears in the vaginal wall, thereby allowing the transmission of the bacterial toxin. The inventors of U.S. Pat. No. 6,488,952 and the present invention are the same. U.S. Pat. No. 6,488,952 makes broad claims on the system's ability to: (1) control exudate flow and (2) resist microbial growth. The present invention clarifies the utilities: (1) for absorption of body fluids and exudate control including synergistic combinations and (2) for an anti-infective agent including synergistic combinations.

### SUMMARY OF THE INVENTION

Therefore, it is an object of the present invention to provide a liquid crystal system and combination liquid crystal, multiparticulate system used to provide for absorption of body fluids and hemostasis, with and without synergistic compound(s) or active drug(s), at 0 to 90% polar, semi-polar, and/or nonpolar solvent content, within or upon body tissues for human or veterinary applications.

It is another object of the present invention to provide absorption & partial clotting of menstrual or otherwise bloody discharge within, upon or in close proximity to female genitalia.

It is another object of the present invention to provide exudate absorption & hemostasis for postpartum, post-op or general feminine reproductive tract bleeding.

It is another object of the present invention to provide exudate absorption & hemostasis for internal and external bleeding resulting from trauma, hemorrhage, injury, disease or surgical procedures.

It is another object of the present invention to provide exudate absorption & hemostasis for internal and external dental or gingival bleeding resulting from trauma, hemorrhage, injury, disease or dental procedures.

It is another object of the present invention to provide exudate absorption & hemostasis for internal and external bleeding resulting from trauma, hemorrhage, injury, disease or surgical procedures of hemorrhoids and anal fissures

It is another object of the present invention to provide a liquid crystal system and combination liquid crystal, multiparticulate system used to provide a moisture balance within or upon body tissues by partial absorption and partial evaporation of body fluids, with and without synergistic compound(s) or active drug(s), at 0 to 90% polar, semi-polar, and/or nonpolar solvent content for human or veterinary applications.

It is another object of the present invention to provide a liquid crystal system and combination liquid crystal, multiparticulate system used to provide a moisture balance within or upon body tissues to speed healing rates of internal and external wounds and/or burns resulting from trauma, hemorrhage, injury, disease or surgical procedures.

It is another object of the present invention to provide a liquid crystal system and combination liquid crystal, multiparticulate system used to provide a moisture balance within or upon body tissues to improve the cosmetics of normal and hypertrophic scars resulting from trauma, disease or surgical procedures.

It is another object of the present invention to provide a liquid crystal system and combination liquid crystal, multiparticulate system used to provide a moisture balance within or upon tissues to provide lubrication and treatment of hemorrhoids and anal fissures before and after surgical intervention.

It is another object of the present invention to provide a liquid crystal system and combination liquid crystal, multiparticulate system used to provide a moisture balance within or upon body tissues for dental treatments and procedures.

It is another object of the present invention to provide a liquid crystal system and combination liquid crystal, multiparticulate system used as an anti-infective agent, with and without synergistic compound(s) or active drug(s), at 0 to 90% polar, semi-polar, and/or nonpolar solvent content, within or upon body tissues for human or veterinary applications.

It is another object of the present invention to provide a liquid crystal system and combination liquid crystal, multiparticulate system used as an anti-infective agent for internal and external wounds and burns wounds or burns resulting from trauma, hemorrhage, injury, disease or surgical procedures.

It is another object of the present invention to provide a liquid crystal system and combination liquid crystal, multiparticulate system used as an anti-infective agent to treat hemorrhoids and anal fissures before and after surgical intervention.

It is another object of the present invention to provide a liquid crystal system and combination liquid crystal, multiparticulate system used as an anti-infective agent to treat dental wounds resulting from trauma, hemorrhage, injury, disease or surgical/dental procedures.

It is another object of the present invention to provide a liquid crystal system and combination liquid crystal, multiparticulate system used as an anti-infective agent coating for medical device hardware intended for implantation into the body.

It is another object of the present invention to provide a liquid crystal system and combination liquid crystal, multiparticulate system used as an anti-infective agent lubricant for the prevention of sexually transmitted diseases.

It is another object of the present invention to provide a liquid crystal system and combination liquid crystal, multiparticulate system used as a lubricant for the insertion of and removal of medical or surgical devices into or from body cavities or orifices.

It is another object of the present invention to provide a liquid crystal system and combination liquid crystal, multiparticulate system used in combination with commercial absorbent products such as feminine hygiene articles, bandages and surgical aids.

It is another object of the present invention to provide a liquid crystal system and combination liquid crystal, multiparticulate system adapted for utilization as an inherent Thrombin inhibitor for the treatment of malignant tumors.

It is another object of the present invention to provide a liquid crystal system and combination liquid crystal, multiparticulate system adapted for utilization as an inherent neuroprotective agent.

It is another object of the present invention to provide a liquid crystal system and combination liquid crystal, multiparticulate system adapted for utilization as an inherent vascular plug.

It is another object of the present invention to provide a liquid crystal system and combination liquid crystal, multiparticulate system adapted for utilization as an injection for cosmetic and functional surgical augmentations.

Briefly described according to one embodiment of the present invention, a semisolid system and combination semisolid, multiparticulate system for controlling biological fluids is provided, comprising a body fluid control composition adapted to be utilized both as an independent mixture for direct application to body tissue for human or veterinary application and to be incorporated with catamenial absorption products, medical/surgical absorbent articles, and related products.

The body fluid control composition is adapted to facilitate absorption of body fluids, promote clotting of blood, promote the establishment of hemostasis or provide sealant properties to exposed or altered tissues. The body fluid control composition is independently delivered to a medical treatment site via injection, irrigation, stream, or spray systems. Alternatively, the body fluid control composition is incorporated for use as a component with a selected absorbent product or medical/surgical article.

The body fluid control composition is defined of a lipid component that may further incorporate synergistic compounds to alter the viscosity or consistency of the system or to enhance the local effects of the system and active medicaments to provide local or systemic therapeutic effects.

A first alternative semisolid, lipid-based system and combination lipid, multiparticulate system is provided, comprising a body fluid moisture equilibrator composition adapted to provide moisture balance within or upon bodily tissues by partial absorption and partial evaporation of bodily fluids for human or veterinary application, thereby accelerating the healing rates of internal and external wounds or burns resulting from trauma, hemorrhage, injury, disease, dental or surgical procedures, as well as controlling menstrual discharge, improving the cosmetics of normal and hypertrophic scars, lubricating and treating hemorrhoids and anal fissures. The body fluid moisture equilibrator composition is adapted to function both independently as a separate liquid or semisolid lipid mixture for direct application, and conjunctively with standard, commercially available medical, surgical, and catamenial absorbent articles.

A second alternative lipid-based system and combination lipid, multiparticulate system is provided, comprising an absorbent, anti-infective composition with inherent properties as an anti-infective agent for human or veterinary application which eliminate or inhibits the progression of infection caused by bacteria, viruses, protozoa, fungi, yeast, and analogous causative agents of infectious diseases of internal and external wounds or burns resulting from trauma, hemorrhage, injury, disease, dental or surgical procedures, as well as to treat hemorrhoids and anal fissures before and after surgical intervention, to coat medical device hardware intended for implantation into the body, and to serve as a lubricant for prevention of sexually transmitted diseases. The absorbent, anti-infective composition is adapted to function both independently as a separate liquid or semisolid lipid mixture for direct application, and conjunctively with standard medical and surgical articles, prophylactic devices and related medical products.

### DESCRIPTION OF THE PREFERRED EMBODIMENT

### 1. Detailed Description of the Invention

A liquid or semisolid, lipid-based system and combination lipid, multiparticulate system for controlling biological fluids is provided, according to the present invention, comprising a body fluid control composition adapted to be utilized both as an independent mixture for direct application to body tissue for human or veterinary application and to be incorporated with a standard and envisioned medical, surgical or catamenial absorption articles for absorption of body fluids and hemostasis. The lipid-based, biological fluid control system is adapted and intended to be incorporated for use with and encompass medical dressing articles and comparable medical absorbent products as will be described in greater detail below.

A. Absorbent, Hemostatic, Antibacterial Tampon

The body fluid control composition of the preferred embodiment of the present invention comprises a hydrophilic or lipophilic component capable of forming semisolid or solid, ordered or disordered, three dimensional systems or structures upon contact with bodily fluids. Preferably, the hydrophilic or lipophilic component is capable of forming a hydrogel or liquid crystalline system. More preferably, the lipid component defined as being greater than 40% fatty acid, fatty acid monoester, fatty acid diester, or fatty acid triester alone or in combination thereby forming a lipid composition. Most preferably, the lipid composition comprises 15.0%-100% by weight of glyceryl monooleate (GMO), wherein a balance of lipid composition is a polar solvent such as water or a physiologic buffered aqueous system or alternatively a semipolar solvent or a nonpolar solvent. Specific solvents include, but are not limited to alcohol(s), polyethylene glycol(s), propylene glycol, and polypropylene glycol. The lipid composition has a solvent content defined at 0%-90% polar, semi-polar, or nonpolar.

The lipid composition, as incorporated with a standard feminine tampon, provides a sanitary catamenial article operable to facilitate absorption and partial clotting of menstrual or otherwise bloody discharge upon contact upon or proximal to female genitalia. Specifically, the standard feminine tampon is defined as including an absorbent material and a liquid-permeable outer covering. The absorbent material comprises rayon fibers formed into a fibrous web. The fibrous web is aggregated into a tubular ribbon, wherein tubular ribbon is thereafter covered in a nonwoven, liquid-permeable fabric made from heat-fusible fibers. Edges of the nonwoven fabric are overlapped and heat-treated to form a seal. The covered tubular ribbon is cut into blanks, whereupon a rayon withdrawal string is pierced and looped through each blank. Each blank is compressed in the manner recognized in the art. An example of a standard, commercially available catamenial absorption article, more specifically, feminine tampon is illustrated in FIG. 1.

The lipid composition may incorporate synergistic compound(s) and active medicament(s), wherein the synergistic compound(s) includes but is not limited to physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, hyaluronic acid and hyaluronic acid derivatives, hyaluronic acid and hyaluronic acid derivatives, cellulose and cellulose derivatives, collagen, solid or solubilized chitosan derivative(s), hydrogel forming agent(s), hydrocolloid forming agent(s), alginic acid derivative(s), oleaginous ointment base(s), absorbent ointment base(s), emulsion ointment base(s), astringent agent(s), vasoconstrictor(s), protein(s) and blood products comprising platelets, prothrombin, thrombin, fibrinogen or clotting factors and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; enzymes and enzyme inhibitors. The oleaginous ointment bases comprise petrolatum, white petrolatum, waxes, cetyl esters wax, oleic acid, olive oil, and spermaceti. The absorbent ointment bases comprise lanolin, hydrophilic petrolatum, hydroxystearin sulfate or components thereof. The emulsion ointment bases comprise cetyl alcohol, stearic acid, hydrophilic ointment, cold cream or components thereof. For purposes of this disclosure, the term “hydrogel” means a substance resulting in a solid, semisolid, pseudoplastic, or plastic structure containing a necessary solvent component to produce a gelatinous or jelly-like mass. The hydrogel incorporates and retains significant amounts of a polar solvent(s) such as water or a semipolar solvent(s) such as ethanol. The term “liquid crystalline” means a substance resulting in an amphiphilic or lipophilic spontaneously forming solid, semisolid, pseudoplastic, or plastic structure containing a necessary solvent component or system to produce a structurally ordered system. Hydrogel forming agent(s) includes but is not limited to hypromellose, alginic acid and derivatives, glycerol monooleate, and hyaluronic acid and derivatives. Liquid crystalline forming agents include but are not limited to glycerol monooleate, glycerol monostearate, glycerol monolaurate, glycerol dilaurate, glyceryl monocaprylate, glyceryl caprate, and glyceryl monopalmitate.

The term “hydrolloid”, as defined for purposes of this disclosure, means water-swellable polymers capable of absorbing large quantities of fluids. Such polymers include but are not limited to polysaccharides, hydroxypropyl cellulose, cationic types including polyvinyl pyridine and N,N-dimethylaminoethyl or N,N-diethylaminopropyl acrylates and methacrylates, and respective quaternary salts thereof, and nonionic types including polyvinyl alcohol and polyvinyl ethers.

Alternatively, the feminine tampon is envisioned as being incorporated with a fluid control film component to facilitate delivery of active medicaments/synergistic compounds to bodily tissue.

The lipid composition with synergistic and medicament components provides a mixture being operable to facilitate absorption of body fluids, hemostasis, independently as a semisolid, gel, or liquid lipid mixture, or as incorporated with a standard feminine tampon upon contact within, upon, or proximal to female genitalia. A further advantage provided by the absorbent, hemostatic, antibacterial tampon is to facilitate maintenance of optimal moisture balance upon or within bodily tissues conjunctively with exudates or discharge control capability.

In order to facilitate incorporation of the lipid composition to the feminine tampon, the lipid composition is applied uniformly to outer surfaces of the feminine tampon via hot-melt coating method in a manner so as to ensure absorptive capacity of the feminine tampon is not exceeded. The resulting product is an absorbent, hemostatic, antibacterial tampon which functions to facilitate absorption and partial clotting of menstrual or otherwise bloody discharge within the female genitalia, as well as, upon or in close proximity thereto, and further functions to inhibit the production of toxic shock syndrome toxin-1 by Staphylococcus aureus bacteria.

Alternative methods for incorporating the lipid composition to a selected article includes general liquid coating by spray, dip, knife spread or cast transfer methodology.

B. First Alternative Absorbent, Hemostatic, Antibacterial Tampon

In order to provide exudate absorption and hemostasis for postpartum or general feminine reproductive tract bleeding, a first alternative absorbent, hemostatic, antibacterial tampon is disclosed and described hereinbelow. The first alternative absorbent, hemostatic, antibacterial tampon incorporates a unique combination of elements so as to provide a sanitary article being particularly operable to facilitate exudate absorption and hemostasis for postpartum hemorrhaging, post-operative, or general feminine reproductive tract bleeding. The first alternative absorbent, hemostatic, antibacterial tampon comprises a body fluid control composition adapted for incorporation with a standard feminine tampon as described hereinabove, and thus such standard feminine tampon is intended to be applied for use with this embodiment.

The body fluid control composition of the preferred embodiment of the present invention comprises a hydrophilic or lipophilic component capable of forming semisolid or solid, ordered or disordered, three dimensional systems or structures upon contact with bodily fluids. Preferably, the hydrophilic or lipophilic component is capable of forming a hydrogel or liquid crystalline system. More preferably, the lipid component defined as being greater than 40% fatty acid, fatty acid monoester, fatty acid diester, or fatty acid triester alone or in combination thereby forming a lipid composition. Most preferably, the lipid composition comprises 15.0%-100% by weight of glyceryl monooleate (GMO), wherein a balance of lipid composition is a polar solvent such as water or a physiologic buffered aqueous system or alternatively a semipolar solvent or a nonpolar solvent. Specific solvents include, but are not limited to alcohol(s), polyethylene glycol(s), propylene glycol, and polypropylene glycol. The lipid composition has a solvent content defined at 0%-90% polar, semi-polar, or nonpolar.

The lipid composition as an independent semisolid, gel, or liquid lipid mixture, or as incorporated with the standard feminine tampon, is operable to facilitate absorption of body fluids, hemostasis for postpartum, post-operative or general feminine reproductive tract bleeding upon contact within, upon, or proximal to female genitalia.

The lipid composition may incorporate synergistic compound(s) and active medicament(s), wherein the synergistic compound(s) includes but is not limited to physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, hyaluronic acid and hyaluronic acid derivatives, hyaluronic acid and hyaluronic acid derivatives, cellulose and cellulose derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes and enzyme inhibitors. The blood products comprise platelets, prothrombin, and fibrinogen.

Alternatively, the standard feminine tampon is envisioned as being incorporated with a fluid control film component to facilitate delivery of active medicaments to bodily tissue.

The lipid composition with synergistic and medicament components provides a mixture being operable to facilitate absorption of body fluids, hemostasis for postpartum, post-operative or general feminine reproductive tract bleeding as incorporated with the standard feminine tampon upon contact within, upon, or proximal to female genitalia.

In order to facilitate incorporation of the lipid composition to the standard feminine tampon, the lipid composition is applied uniformly to outer surfaces of the feminine tampon via hot-melt coating method in a manner so as to ensure absorptive capacity of the feminine tampon is not exceeded. The resulting product is a first alternative absorbent, hemostatic, antibacterial tampon which functions to facilitate bodily fluid absorption, hemostasis for postpartum, post-operative or general feminine reproductive tract bleeding within the female genitalia, as well as, upon or in close proximity thereto, and further functions to inhibit the production of toxic shock syndrome toxin-1 by Staphylococcus aureus bacteria.

Alternative methods for incorporating the lipid composition to a selected feminine article includes general liquid coating by spray, dip, knife spread or cast transfer methodology.

It is envisioned that the lipid composition as an independent mixture may be delivered to an effected area via injection, irrigation, stream, or spray.

C. Absorbent, Hemostatic, Antibacterial Feminine Menstrual Pad

A body fluid control composition adapted specifically for incorporation with a standard, commercially available feminine menstrual pad, napkin, or related feminine article is disclosed to provide a catamenial absorption and hemostatic article. The standard feminine menstrual pad is more specifically defined as comprising a liquid-permeable upper side, an absorbency core underlying the liquid-permeable upper side, a liquid-impermeable underside, and a barrier layer interposed between the absorbency core and the liquid-impermeable underside. The liquid-permeable upper side of the standard feminine menstrual pad is intended to be directed towards the user during use, and the liquid-impermeable underside is intended to be directed away from the user during use. The liquid-permeable upper side, the absorbency core, the barrier layer, and the liquid-impermeable underside are bonded so as to form a seal along a periphery of the standard feminine menstrual pad. The seal is produced by a method selected from the group consisting of thermal welding, ultrasonic welding, mechanical crimping, and adhesive bonding. The standard feminine menstrual pad further comprises a peel strip on the rear side of the liquid-impermeable underside covering a layer of adhesive. A pair of securing wings is provided wherein each extends laterally of a central portion of pad at opposed sides thereof. An example of a standard, commercially available catamenial absorption article, more specifically menstrual pad, is illustrated in FIG. 2.

The body fluid control composition of the preferred embodiment of the present invention comprises a hydrophilic or lipophilic component capable of forming semisolid or solid, ordered or disordered, three dimensional systems or structures upon contact with bodily fluids. Preferably, the hydrophilic or lipophilic component is capable of forming a hydrogel or liquid crystalline system. More preferably, the lipid component defined as being greater than 40% fatty acid, fatty acid monoester, fatty acid diester, or fatty acid triester alone or in combination thereby forming a lipid composition. Most preferably, the lipid composition comprises 15.0%-100% by weight of glyceryl monooleate (GMO), wherein a balance of lipid composition is a polar solvent such as water or a physiologic buffered aqueous system or alternatively a semipolar solvent or a nonpolar solvent. Specific solvents include, but are not limited to alcohol(s), polyethylene glycol(s), propylene glycol, and polypropylene glycol. The lipid composition has a solvent content defined at 0%-90% polar, semi-polar, or nonpolar.

The lipid composition, as incorporated with a standard feminine menstrual pad, is operable to facilitate absorption of body fluids, partial clotting of menstrual or otherwise bloody discharge, and the inhibition of the production of toxic shock syndrome toxin-1 by Staphylococcus aureus bacteria upon contact within, upon, or proximal to female genitalia.

The lipid composition may incorporate synergistic compound(s) and active medicament(s), wherein the synergistic compound(s) includes but is not limited to physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, hyaluronic acid and hyaluronic acid derivatives, cellulose and cellulose derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes and enzyme inhibitors. The blood products comprise platelets, prothrombin, and fibrinogen.

Alternatively, the feminine menstrual pad is envisioned as being incorporated with a fluid control film component to facilitate delivery of active medicaments/synergistic compounds to bodily tissue.

The lipid composition with synergistic and medicament components provides a mixture being operable to facilitate absorption of body fluids, partial clotting of menstrual or otherwise bloody discharge, and the inhibition of the production of toxic shock syndrome toxin-1 by Staphylococcus aureus bacteria independently as a semisolid, gel, or liquid lipid mixture, or as incorporated with a standard feminine menstrual pad upon contact within, upon, or proximal to female genitalia.

In order to facilitate incorporation of the lipid composition to the feminine menstrual pad, the lipid composition is applied uniformly to the outer surface of the liquid-permeable upper side of the feminine menstrual pad via hot-melt coating method in a manner so as to ensure absorptive capacity of the pad is not exceeded. Alternative methods for incorporating the lipid composition to a selected article includes general liquid coating by spray, dip, knife spread or cast transfer methodology. The resulting product provides an absorbent antibacterial feminine menstrual pad which functions to facilitate absorption and partial clotting of menstrual or otherwise bloody discharge upon or proximal to female genitalia, and further functions to inhibit the production of toxic shock syndrome toxin-1 by Staphylococcus aureus bacteria.

A further advantage provided by the absorbent, antibacterial feminine menstrual pad is to facilitate maintenance of optimal moisture balance upon or proximal to bodily tissues, particularly female genitalia, in a conjunctive manner with its menstrual discharge control capability.

D. First Alternative Absorbent, Hemostatic, Antibacterial Feminine Menstrual Pad

In order to provide exudate absorption and hemostasis for postpartum or general feminine reproductive tract bleeding, a first alternative absorbent, hemostatic, antibacterial feminine menstrual pad or feminine napkin is disclosed and described hereinbelow. The first alternative absorbent, hemostatic, antibacterial feminine menstrual pad incorporates a unique combination of elements so as to provide a sanitary article such as a menstrual pad, a feminine napkin and the like being particularly operable to facilitate exudate absorption and hemostasis for postpartum hemorrhaging, post-operative or general feminine reproductive tract bleeding. The first alternative absorbent, hemostatic, antibacterial feminine menstrual pad comprises a body fluid control composition adapted for incorporation with a standard feminine menstrual pad as described hereinabove, and thus such standard feminine menstrual pad is intended to be applied for use with this embodiment.

The body fluid control composition of the preferred embodiment of the present invention comprises a hydrophilic or lipophilic component capable of forming semisolid or solid, ordered or disordered, three dimensional systems or structures upon contact with bodily fluids. Preferably, the hydrophilic or lipophilic component is capable of forming a hydrogel or liquid crystalline system. More preferably, the lipid component defined as being greater than 40% fatty acid, fatty acid monoester, fatty acid diester, or fatty acid triester alone or in combination thereby forming a lipid composition. Most preferably, the lipid composition comprises 15.0%-100% by weight of glyceryl monooleate (GMO), wherein a balance of lipid composition is a polar solvent such as water or a physiologic buffered aqueous system or alternatively a semipolar solvent or a nonpolar solvent. Specific solvents include, but are not limited to alcohol(s), polyethylene glycol(s), propylene glycol, and polypropylene glycol. The lipid composition has a solvent content defined at 0%-90% polar, semi-polar, or nonpolar.

The lipid composition as an independent semisolid, gel, or liquid lipid mixture, or as incorporated with the standard feminine menstrual pad, is operable to facilitate absorption of body fluids, hemostasis for postpartum, post-operative, or general feminine reproductive tract bleeding, and the inhibition of the production of toxic shock syndrome toxin-1 by Staphylococcus aureus bacteria upon contact upon or proximal to female genitalia.

The lipid composition may incorporate synergistic compound(s) and active medicament(s), wherein the synergistic compound(s) includes but is not limited to physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, hyaluronic acid and hyaluronic acid derivatives, cellulose and cellulose derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes and enzyme inhibitors. The blood products comprise platelets, prothrombin, and fibrinogen.

The active medicament(s) to be incorporated into the lipid composition includes but is not limited to anti-infective agents, a sulfa derivative, an anti-inflammatory drug, a steroid derivative, a non-steroidal anti-inflammatory derivative, and an enzyme inhibitor. The anti-infective agents comprise an antibiotic, an antifungal, and an antiviral. Alternatively, the standard feminine menstrual pad is envisioned as being incorporated with a fluid control film component to facilitate delivery of active medicaments to bodily tissue.

The lipid composition with synergistic and medicament components provides an independent semisolid, gel, or liquid lipid mixture or as incorporated with the standard feminine menstrual pad being operable to facilitate absorption of body fluids, hemostasis for postpartum, post-operative or general feminine reproductive tract bleeding, and the inhibition of the production of toxic shock syndrome toxin-1 by Staphylococcus aureus bacteria upon contact upon or proximal to female genitalia.

In order to facilitate incorporation of the lipid composition to the standard feminine menstrual pad, the lipid composition is applied uniformly to the outer surface of the liquid-permeable upper side of the menstrual pad via hot-melt coating method in a manner so as to ensure absorptive capacity of the pad is not exceeded. Alternative methods for incorporating the lipid composition to a selected feminine article includes general liquid coating by spray, dip, knife spread or cast transfer methodology. The resulting product is a first alternative absorbent, hemostatic, antibacterial feminine menstrual pad which functions to facilitate bodily fluid absorption, and hemostasis for postpartum, post-operative or general feminine reproductive tract bleeding upon or proximal to the female genitalia, and wherein first alternative absorbent, hemostatic, antibacterial feminine menstrual pad further functions to inhibit the production of toxic shock syndrome toxin-1 by Staphylococcus aureus bacteria.

It is envisioned that the lipid composition as an independent mixture may be delivered to an effected area via injection, irrigation, stream, or spray.

E. Absorbent, Hemostatic Secondary Sponge or Dressing

In order to provide exudate absorption and hemostasis for postpartum, post-operative or general feminine reproductive tract bleeding, an absorbent, hemostatic, secondary sponge or wound dressing is disclosed and described hereinbelow. For purposes of this particular application, other sanitary medical articles including but not limited to swellable sponges, gauzes, fibrotic packings, and compresses are fully within the scope of this embodiment. The absorbent, hemostatic, secondary sponge or dressing incorporates a unique combination of elements so as to provide a sanitary article being particularly operable to facilitate exudate absorption and hemostasis for postpartum, post-operative hemorrhaging or general feminine reproductive tract bleeding. The absorbent, hemostatic, secondary sponge or dressing comprises a body fluid control composition adapted for incorporation with a standard, commercially available secondary sponge or dressing commonly known and associated in the medical/surgical environment. A descriptive example of an illustrative medical absorbent article suitable for use in the present invention is described in U.S. Pat. No. 4,798,201, which is herein incorporated by reference.

The body fluid control composition of the preferred embodiment of the present invention comprises a hydrophilic or lipophilic component capable of forming semisolid or solid, ordered or disordered, three dimensional systems or structures upon contact with bodily fluids. Preferably, the hydrophilic or lipophilic component is capable of forming a hydrogel or liquid crystalline system. More preferably, the lipid component defined as being greater than 40% fatty acid, fatty acid monoester, fatty acid diester, or fatty acid triester alone or in combination thereby forming a lipid composition. Most preferably, the lipid composition comprises 15.0%-100% by weight of glyceryl monooleate (GMO), wherein a balance of lipid composition is a polar solvent such as water or a physiologic buffered aqueous system or alternatively a semipolar solvent or a nonpolar solvent. Specific solvents include, but are not limited to alcohol(s), polyethylene glycol(s), propylene glycol, and polypropylene glycol. The lipid composition has a solvent content defined at 0%-90% polar, semi-polar, or nonpolar.

The lipid composition provides a mixture being operable to facilitate absorption of body fluids, hemostasis for postpartum, post-operative or general feminine reproductive tract bleeding independently as a semisolid, gel, or liquid lipid mixture, or as incorporated with a standard secondary sponge or dressing upon contact upon or proximal to female genitalia.

The lipid composition may incorporate synergistic compound(s) and active medicament(s), wherein the synergistic compound(s) includes but is not limited to physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, hyaluronic acid and hyaluronic acid derivatives, cellulose and cellulose derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes and enzyme inhibitors. The blood products comprise platelets, prothrombin, and fibrinogen.

Alternatively, the standard sponge or dressing is envisioned as being incorporated with a fluid control film component to facilitate delivery of active medicaments to bodily tissue.

The lipid composition with synergistic and medicament components provides a mixture being operable to facilitate absorption of body fluids, hemostasis for postpartum, post-operative or general feminine reproductive tract bleeding independently, or as incorporated with a standard secondary sponge or dressing upon contact upon or proximal to female genitalia.

In order to facilitate incorporation of the lipid composition to the standard sponge or dressing, the lipid composition is applied uniformly to an outer surface thereof via hot-melt coating method in a manner so as to ensure absorptive capacity of the sponge or dressing is not exceeded. Alternative methods for incorporating the lipid composition to a selected medical article includes general liquid coating by spray, dip, knife spread or cast transfer methodology.

It is envisioned that the lipid composition as an independent mixture may be delivered to an effected area via injection, irrigation, stream, or spray.

F. Exudate-Absorbent, Hemostatic Tissue Sealant

In order to facilitate exudate absorption and hemostasis for internal and external bleeding resulting from trauma, hemorrhage, injury, disease or surgical procedures, an exudate-absorbent, hemostatic tissue sealant is disclosed and described hereinbelow. The exudate-absorbent, hemostatic tissue sealant incorporates a unique combination of elements so as to provide both an independent lipid mixture and a sanitary medical article being particularly operable to facilitate exudate absorption and hemostasis for internal and external bleeding. The exudate-absorbent, hemostatic tissue sealant comprises a body fluid control composition specifically adapted for incorporation with the standard secondary sponge or wound dressing described above, and is intended to be applied for use with this embodiment. For purposes of this particular application, other sanitary medical articles including but not limited to swellable sponges, fibrotic packings, adhesive bandages, and compresses are also fully within the scope of this embodiment.

The body fluid control composition of the preferred embodiment of the present invention comprises a hydrophilic or lipophilic component capable of forming semisolid or solid, ordered or disordered, three dimensional systems or structures upon contact with bodily fluids. Preferably, the hydrophilic or lipophilic component is capable of forming a hydrogel or liquid crystalline system. More preferably, the lipid component defined as being greater than 40% fatty acid, fatty acid monoester, fatty acid diester, or fatty acid triester alone or in combination thereby forming a lipid composition. Most preferably, the lipid composition comprises 15.0%-100% by weight of glyceryl monooleate (GMO), wherein a balance of lipid composition is a polar solvent such as water or a physiologic buffered aqueous system or alternatively a semipolar solvent or a nonpolar solvent. Specific solvents include, but are not limited to alcohol(s), polyethylene glycol(s), propylene glycol, and polypropylene glycol. The lipid composition has a solvent content defined at 0%-90% polar, semi-polar, or nonpolar.

The lipid composition provides a mixture being operable to facilitate absorption of body fluids and hemostasis for internal and external bleeding independently as a semisolid, gel, or liquid lipid mixture, or as incorporated with a secondary sponge, dressing or other absorbent article.

The lipid composition, during an initial liquid phase thereof, may incorporate synergistic compound(s) and active medicament(s) in the liquid or solid state as a simple mixture with lipid composition, or as independent granulates, agglomerates or coated particles. The synergistic compound(s) includes but is not limited to physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, hyaluronic acid and hyaluronic acid derivatives, cellulose and cellulose derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes and enzyme inhibitors. The blood products comprise platelets, prothrombin, and fibrinogen. The synergistic compounds are operable as primary or adjunctive therapy or as structures to enhance or otherwise alter the structure, consistency, or function of the lipid composition.

Alternatively, the standard sponge or dressing is envisioned as being incorporated with a fluid control film component to facilitate delivery of active medicaments to bodily tissue.

The granulates, agglomerates, or coated particles are produced by a method known in the art consisting of a fluid bed processes, a low shear processes, and a high shear processes, precipitation, or coacervation.

The lipid composition with synergistic and medicament components provides a mixture being operable to facilitate absorption of body fluids and hemostasis for internal and external bleeding independently as a semisolid, gel, or liquid lipid mixture, or as incorporated with a secondary dressing, bandage, or other absorbent article.

In order to facilitate delivery of the lipid composition as an independent semisolid, gel, or liquid lipid mixture, independent lipid composition is delivered or transmitted as a suspension or solution thereof to medical treatment site or effected area via injection, irrigation, stream, or spray.

In order to facilitate delivery of the lipid composition as incorporated with a standard secondary dressing, bandage or other absorbent article, the lipid composition is applied uniformly to outer surfaces of the dressing via hot-melt coating method in a manner so as to ensure absorptive capacity of the dressing is not exceeded. Alternative methods for incorporating the lipid composition to a selected medical article includes general liquid coating by spray, dip, knife spread or cast transfer methodology. The resulting product according the aforementioned respective means of transmission is an independent lipid composition and an exudate-absorbent, hemostatic secondary sponge or dressing which function to facilitate exudate absorption and hemostasis for internal and external bleeding resulting from trauma, hemorrhage, injury, disease, or surgical procedures.

It is envisioned that the wound dressing is used as a transient structure for absorption of excess blood within a wound with the lipid composition providing the primary action of establishing hemostasis as would be necessary within a surgical or traumatic wound or a nasal plug for Epistaxis. It is further envisioned that a translucent viewing area is provided along a portion of a standard secondary dressing or bandage in order to allow for direct visual observation of wound healing progress.

G. First Alternative Exudate-Absorbent, Hemostatic Tissue Sealant

In order to facilitate exudate absorption and hemostasis for internal and external dental or gingival bleeding resulting from trauma, hemorrhage, injury, disease, or dental procedures, a first alternative exudate-absorbent, hemostatic tissue sealant is disclosed and described hereinbelow. The first alternative exudate-absorbent, hemostatic tissue sealant incorporates a unique combination of elements so as to provide a sanitary medical article being particularly operable to facilitate exudate absorption and hemostasis for internal and external dental bleeding. The first alternative exudate-absorbent, hemostatic tissue sealant comprises a body fluid control composition specifically adapted for independent use as a semisolid, gel or liquid lipid mixture, adapted for incorporation with gauze, bandages or wound dressings commonly known and associated in the medical environment, or adapted for use as a component of a dental kit. For purposes of this particular application, other sanitary medical articles including but not limited to swellable sponges, fibrotic packings, and compresses are also fully within the scope of this embodiment.

The body fluid control composition of the preferred embodiment of the present invention comprises a hydrophilic or lipophilic component capable of forming semisolid or solid, ordered or disordered, three dimensional systems or structures upon contact with bodily fluids. Preferably, the hydrophilic or lipophilic component is capable of forming a hydrogel or liquid crystalline system. More preferably, the lipid component defined as being greater than 40% fatty acid, fatty acid monoester, fatty acid diester, or fatty acid triester alone or in combination thereby forming a lipid composition. Most preferably, the lipid composition comprises 15.0%-100% by weight of glyceryl monooleate (GMO), wherein a balance of lipid composition is a polar solvent such as water or a physiologic buffered aqueous system or alternatively a semipolar solvent or a nonpolar solvent. Specific solvents include, but are not limited to alcohol(s), polyethylene glycol(s), propylene glycol, and polypropylene glycol. The lipid composition has a solvent content defined at 0%-90% polar, semi-polar, or nonpolar.

The lipid composition provides a mixture being operable to facilitate exudate absorption and hemostasis for internal and external dental or gingival bleeding either independently as a semisolid, gel, or liquid lipid mixture, or as incorporated with a standard adhesive bandage or wound dressing, or as a component of the dental kit.

The lipid composition may incorporate synergistic compound(s) and active medicament(s), wherein the synergistic compound(s) includes but is not limited to physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, hyaluronic acid and hyaluronic acid derivatives, cellulose and cellulose derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes and enzyme inhibitors; a gingivitis agent; a periodontal agent; a fluoride derivative(s); triclosan or carbamide peroxide. The blood products comprise platelets, prothrombin, and fibrinogen.

Alternatively, the standard adhesive bandage or wound dressing is envisioned as being incorporated with a fluid control film component to facilitate delivery of active medicaments to bodily tissue.

In order to facilitate delivery of the lipid composition as an independent semisolid, gel, or liquid lipid mixture, the independent lipid composition is delivered or transmitted as a suspension or solution thereof to medical treatment site or effected area via injection, irrigation, stream, or spray.

In order to facilitate incorporation of the lipid composition to a gauze, bandage or wound dressing, the lipid composition is applied uniformly to a wound-contacting surface thereof by a method, such as hot-melt coating, being suitable to facilitate uniform application and adherence by lipid composition to wound-contacting surface, and in a manner which ensures absorptive capacity of the adhesive bandage and wound dressing is not exceeded. Alternative methods for incorporating the lipid composition to a selected medical article includes general liquid coating by spray, dip, knife spread or cast transfer methodology.

The lipid composition with synergistic and medicament components provides a mixture being operable to facilitate exudate absorption and hemostasis for internal and external dental or gingival bleeding either independently, or as incorporated with a gauze, bandage and wound dressing.

It is envisioned that a translucent viewing area is provided along a portion of a standard adhesive bandage and wound dressing in order to allow for direct visual observation of wound healing progress.

H. Second Alternative Exudate-Absorbent, Hemostatic Tissue Sealant

In order to facilitate exudate absorption and hemostasis for internal and external bleeding resulting from trauma, hemorrhage, injury, disease or surgical procedures of the gastrointestinal or genitourinary tract with hemorrhoids and anal fissures as non-limiting examples, a second alternative exudate-absorbent, hemostatic tissue sealant is disclosed and described hereinbelow. The second alternative exudate-absorbent, hemostatic tissue sealant incorporates a unique combination of elements so as to provide a sanitary medical article being particularly operable to facilitate exudate absorption and hemostasis for internal and external bleeding. The second alternative exudate-absorbent, hemostatic tissue sealant comprises a body fluid control composition for use independently or specifically adapted for incorporation with a secondary sponge, gauze or wound dressing described above, and is intended to be applied for use with this embodiment. For purposes of this particular application, other sanitary medical/surgical articles including but not limited to swellable sponges, fibrotic packings, and compresses are also fully within the scope of this embodiment.

The body fluid control composition of the preferred embodiment of the present invention comprises a hydrophilic or lipophilic component capable of forming semisolid or solid, ordered or disordered, three dimensional systems or structures upon contact with bodily fluids. Preferably, the hydrophilic or lipophilic component is capable of forming a hydrogel or liquid crystalline system. More preferably, the lipid component defined as being greater than 40% fatty acid, fatty acid monoester, fatty acid diester, or fatty acid triester alone or in combination thereby forming a lipid composition. Most preferably, the lipid composition comprises 15.0%-100% by weight of glyceryl monooleate (GMO), wherein a balance of lipid composition is a polar solvent such as water or a physiologic buffered aqueous system or alternatively a semipolar solvent or a nonpolar solvent. Specific solvents include, but are not limited to alcohol(s), polyethylene glycol(s), propylene glycol, and polypropylene glycol. The lipid composition has a solvent content defined at 0%-90% polar, semi-polar, or nonpolar.

The lipid composition provides a mixture being operable to facilitate exudate absorption and hemostasis for internal and external bleeding resulting from trauma, hemorrhage, injury, disease or surgical procedures of the gastrointestinal or genitourinary tract as in the case of hemorrhoids and anal fissures independently as a semisolid, gel, or liquid lipid mixture, or as incorporated with a secondary sponge, gauze or wound dressing.

The lipid composition may incorporate synergistic compound(s) and active medicament(s). The synergistic compound(s) includes but is not limited to physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, hyaluronic acid and hyaluronic acid derivatives, cellulose and cellulose derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes and enzyme inhibitors. The blood products comprise platelets, prothrombin, and fibrinogen.

Alternatively, the standard secondary sponge or wound dressing is envisioned as being incorporated with a fluid control film component to facilitate delivery of active medicaments to bodily tissue.

In order to facilitate delivery of the lipid composition as an independent semisolid, gel, or liquid lipid mixture, the independent lipid composition is delivered or transmitted as a suspension or solution thereof to medical treatment site or effected area via injection, irrigation, stream, or spray.

The lipid composition with synergistic and medicament components provides a mixture being operable to facilitate exudate absorption and hemostasis for internal and external bleeding independently as a semisolid, gel, or liquid lipid mixture, or as incorporated with a secondary sponge, gauze or wound dressing.

In order to facilitate delivery of the lipid composition as an independent semisolid, gel, or liquid lipid mixture, independent lipid composition is delivered or transmitted as a suspension or solution thereof to medical treatment site via aerosol spray or pump spray.

In order to facilitate delivery of the lipid composition as incorporated with a standard secondary sponge or wound dressing, the lipid composition is applied uniformly to outer surfaces of the sponge or dressing via hot-melt coating method in a manner so as to ensure absorptive capacity of the sponge or dressing is not exceeded. Alternative methods for incorporating the lipid composition to a selected medical article includes general liquid coating by spray, dip, knife spread or cast transfer methodology. The resulting product according to both respective means of transmission is an independent lipid composition and an exudate-absorbent, hemostatic secondary sponge or dressing which function to facilitate exudate absorption and hemostasis for internal and external bleeding resulting from trauma, hemorrhage, injury, disease or surgical procedures of the gastrointestinal or genitourinary tract such as in the treatment of hemorrhoids and anal fissures.

### 2. Description of the Alternate Embodiments

An alternative lipid-based system and combination lipid, multiparticulate system is provided, according to an alternate embodiment of the present invention, comprising a body fluid moisture equilibrator composition adapted to provide moisture balance within or upon bodily tissues by partial absorption and partial evaporation of bodily fluids for human or veterinary application, thereby accelerating the healing rates of internal and external wounds or burns resulting from trauma, hemorrhage, injury, disease, or surgical procedures, as well as controlling menstrual discharge, improving the cosmetics of normal and hypertrophic scars, use in gastrointestinal and genitourinary disorders such as lubricating and treating hemorrhoids and anal fissures, and providing treatment in dental procedures and pathology. The body fluid moisture equilibrator composition is adapted to function both independently as a separate lipid mixture, and conjunctively with medical/surgical/feminine articles including, but not limited to bandages, sponges, dressings, tampons, feminine pads, and related medical products described earlier in order to accelerate healing rates of internal and external wounds or burns, in addition to controlling menstrual discharge and improving the cosmetics of normal and hypertrophic scars.

A. Absorbent, Equilibratory Secondary Dressing or Bandage

In order to provide an optimal moisture balance within or upon bodily tissues, more specifically, internal and external wounds or burns when administering medical treatment such as through the application of medical coverings, an absorbent, equilibratory secondary dressing or adhesive bandage is disclosed and described hereinbelow. The absorbent, equilibratory secondary dressing or adhesive bandage incorporates a unique combination of elements so as to provide a sanitary article being particularly operable to facilitate optimal moisture balance within or upon bodily tissues by partial absorption and partial evaporation of bodily fluids, thereby accelerating the healing rates of internal and external wounds or burns. The absorbent, equilibratory secondary dressing or adhesive bandage comprises a body fluid moisture equilibrator composition adapted for incorporation with a standard secondary dressing or adhesive bandage commonly known and associated in the medical/surgical environment as was described above. The earlier described standard, commercially available secondary dressing or adhesive bandage is intended to be applied for use with this embodiment. However, for purposes of this particular application, other sanitary medical/surgical articles including but not limited to wound dressings, swellable sponges, fibrotic packings, and compresses are also fully within the scope of this embodiment.

The body fluid control composition of the preferred embodiment of the present invention comprises a hydrophilic or lipophilic component capable of forming semisolid or solid, ordered or disordered, three dimensional systems or structures upon contact with bodily fluids. Preferably, the hydrophilic or lipophilic component is capable of forming a hydrogel or liquid crystalline system. More preferably, the lipid component defined as being greater than 40% fatty acid, fatty acid monoester, fatty acid diester, or fatty acid triester alone or in combination thereby forming a lipid composition. Most preferably, the lipid composition comprises 15.0%-100% by weight of glyceryl monooleate (GMO), wherein a balance of lipid composition is a polar solvent such as water or a physiologic buffered aqueous system or alternatively a semipolar solvent or a nonpolar solvent. Specific solvents include, but are not limited to alcohol(s), polyethylene glycol(s), propylene glycol, and polypropylene glycol. The lipid composition has a solvent content defined at 0%-90% polar, semi-polar, or nonpolar.

The lipid composition provides a mixture being operable to facilitate optimal moisture balance within or upon internal and external wounds or burns by partial absorption and partial evaporation of bodily fluids, thereby accelerating the healing rates of such wounds or burns independently as a semisolid, gel, or liquid lipid mixture, or as incorporated with a standard secondary dressing or adhesive bandage.

The lipid composition may incorporate synergistic compound(s) and active medicament(s), wherein the synergistic compound(s) includes but is not limited to physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, hyaluronic acid and hyaluronic acid derivatives, cellulose and cellulose derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes and enzyme inhibitors. The blood products comprise platelets, prothrombin, and fibrinogen.

Alternatively, the standard secondary dressing or adhesive bandage is envisioned as being incorporated with a fluid control film component to facilitate delivery of active medicaments to bodily tissue.

In order to facilitate delivery of the lipid composition as an independent semisolid, gel, or liquid lipid mixture, the independent lipid composition is delivered or transmitted as a suspension or solution thereof to medical treatment site or effected area via injection, irrigation, stream, or spray.

In order to facilitate delivery of the lipid composition as incorporated with the standard secondary sponge or wound dressing, the lipid composition is applied uniformly to outer surfaces of the sponge or dressing via hot-melt coating method in a manner so as to ensure absorptive capacity of the sponge or dressing is not exceeded. Alternative methods for incorporating the lipid composition to a selected medical article includes general liquid coating by spray, dip, knife spread or cast transfer methodology.

The lipid composition with synergistic and medicament components provides optimal moisture balance within or upon internal and external wounds or burns by partial absorption and partial evaporation of bodily fluids, thereby accelerating the healing rates of such wounds or burns independently, or as incorporated with a standard secondary dressing or adhesive bandage.

B. Absorbent, Equilibratory Tampon

In order to provide an optimal moisture balance within or upon bodily tissues by partial absorption and partial evaporation of bodily fluids, thereby controlling menstrual discharge within, upon, or in close proximity to female genitalia, an absorbent, equilibratory tampon is disclosed and described hereinbelow. The absorbent, equilibratory tampon incorporates a unique combination of elements so as to provide a sanitary, feminine article being particularly operable to facilitate both optimal moisture balance within, upon, or in close proximity to female genitalia, and menstrual discharge control. The absorbent, equilibratory tampon comprises a body fluid moisture equilibrator composition adapted for incorporation with a standard catamenial absorption article, which includes but is not limited to, a standard feminine tampon, sanitary napkin, menstrual pad, and related catamenial product as described previously above.

The body fluid control composition of the preferred embodiment of the present invention comprises a hydrophilic or lipophilic component capable of forming semisolid or solid, ordered or disordered, three dimensional systems or structures upon contact with bodily fluids. Preferably, the hydrophilic or lipophilic component is capable of forming a hydrogel or liquid crystalline system. More preferably, the lipid component defined as being greater than 40% fatty acid, fatty acid monoester, fatty acid diester, or fatty acid triester alone or in combination thereby forming a lipid composition. Most preferably, the lipid composition comprises 15.0%-100% by weight of glyceryl monooleate (GMO), wherein a balance of lipid composition is a polar solvent such as water or a physiologic buffered aqueous system or alternatively a semipolar solvent or a nonpolar solvent. Specific solvents include, but are not limited to alcohol(s), polyethylene glycol(s), propylene glycol, and polypropylene glycol. The lipid composition has a solvent content defined at 0%-90% polar, semi-polar, or nonpolar.

The lipid composition provides a mixture being operable to facilitate optimal moisture balance within, upon, or in close proximity to female genitalia by partial absorption and partial evaporation of bodily fluids, thereby facilitating menstrual discharge control independently as a semisolid, gel, or liquid lipid mixture, or as incorporated with a standard tampon.

The lipid composition may incorporate synergistic compound(s) and active medicament(s), wherein the synergistic compound(s) includes but is not limited to calcium derivative(s), solid or solubilized chitosan derivative(s), hydrogel forming agent(s), hydrocolloid forming agent(s), alginic acid derivative(s), sulfate derivative(s), potassium derivative(s), phospholipids, oleaginous ointment bases, absorbent ointment bases, emulsion ointment bases, vasoconstrictor, astringent agent(s). The oleaginous ointment bases comprise petrolatum, white petrolatum, waxes, cetyl esters wax, oleic acid; olive oil, and spermaceti. The absorbent ointment bases comprise lanolin, hydrophilic petrolatum, hydroxystearin sulfate or components thereof. The emulsion ointment bases comprise cetyl alcohol, stearic acid, hydrophilic ointment, cold cream or components thereof.

Alternatively, the standard feminine tampon is envisioned as being incorporated with a fluid control film component to facilitate delivery of synergistic compounds and active medicaments to bodily tissue.

In order to facilitate delivery of the lipid composition as incorporated with the standard tampon, the lipid composition is applied uniformly to outer surfaces tampon via hot-melt coating method in a manner so as to ensure absorptive capacity of the tampon is not exceeded. Alternative methods for incorporating the lipid composition to a selected catamenial article includes general liquid coating by spray, dip, knife spread or cast transfer methodology. The lipid composition with synergistic and medicament components provides optimal moisture balance within, upon, or in close proximity to female genitalia, by partial absorption and partial evaporation of bodily fluids, thereby also facilitating menstrual discharge control independently as a semisolid, gel, or liquid lipid mixture, or as incorporated with the standard tampon.

C. Absorbent, Equilibratory Cosmetic Improvement Article

In order to provide an optimal moisture balance within or upon bodily tissues so as to cosmetically improve normal and hypertrophic scars, an absorbent, equilibratory cosmetic improvement article is disclosed and described hereinbelow. The cosmetic improvement article incorporates a unique combination of elements so as to provide an article being particularly operable to facilitate optimal moisture balance within or upon bodily tissues by partial absorption and partial evaporation of bodily fluids, thereby cosmetically improving normal and hypertrophic scars resulting from trauma, disease, or surgical procedures. The absorbent, equilibratory cosmetic improvement article comprises a body fluid moisture equilibrator composition adapted for incorporation with a standard secondary sponge or wound dressing commonly known and associated in the medical/surgical environment. For purposes of this particular application, other sanitary medical/surgical articles including but not limited to adhesive bandages, swellable sponges, fibrotic packings, compresses and related medical articles adapted for scar management use are also fully within the scope of this embodiment.

The body fluid control composition of the preferred embodiment of the present invention comprises a hydrophilic or lipophilic component capable of forming semisolid or solid, ordered or disordered, three dimensional systems or structures upon contact with bodily fluids. Preferably, the hydrophilic or lipophilic component is capable of forming a hydrogel or liquid crystalline system. More preferably, the lipid component defined as being greater than 40% fatty acid, fatty acid monoester, fatty acid diester, or fatty acid triester alone or in combination thereby forming a lipid composition. Most preferably, the lipid composition comprises 15.0%-100% by weight of glyceryl monooleate (GMO), wherein a balance of lipid composition is a polar solvent such as water or a physiologic buffered aqueous system or alternatively a semipolar solvent or a nonpolar solvent. Specific solvents include, but are not limited to alcohol(s), polyethylene glycol(s), propylene glycol, and polypropylene glycol. The lipid composition has a solvent content defined at 0%-90% polar, semi-polar, or nonpolar.

The lipid composition provides a mixture being operable to facilitate optimal moisture balance within or upon normal and hypertrophic scars, thereby improving cosmetically normal and hypertrophic scars independently as a semisolid, gel, or liquid lipid mixture, or as incorporated with the standard secondary sponge or wound dressing.

The lipid composition may incorporate synergistic compound(s) and active medicament(s), wherein the synergistic compound(s) includes but is not limited to onion extract, silicone derivative(s), astringent agent(s), and vasoconstrictor(s).

The active medicament(s) to be incorporated into the lipid composition includes but is not limited to an anti-inflammatory drug, a steroid derivative, a non-steroidal anti-inflammatory derivative, or amino acids, peptides or proteins. Alternatively, the standard secondary sponge or wound dressing is envisioned as being incorporated with a fluid control film component to facilitate delivery of active medicaments to bodily tissue.

In order to facilitate incorporation of the lipid composition to the standard secondary sponge or wound dressing, the lipid composition is applied uniformly to outer surfaces of the sponge or dressing via hot-melt coating method in a manner so as to ensure the absorptive capacity thereof is not exceeded. Alternative methods for incorporating the lipid composition to a selected medical/surgical article includes general liquid coating by spray, dip, knife spread or cast transfer methodology. The lipid composition with synergistic and medicament components provides a mixture being operable to facilitate optimal moisture balance within or upon normal and hypertrophic scars thereby cosmetically improving normal and hypertrophic scars independently as a semisolid, gel, or liquid lipid mixture, or as incorporated with a standard secondary sponge or wound dressing.

It is envisioned that a translucent viewing area is provided along a portion of a standard sponge or wound dressing in order to allow for direct visual observation of wound healing progress.

D. Absorbent, Anti-Infective, Lubricant Product

In order to provide lubrication and treatment of anorectal conditions and pathologies such as hemorrhoids and anal fissures before and after surgical intervention, an absorbent, anti-infective, lubricant product is disclosed and described hereinbelow. The absorbent, anti-infective, lubricant product comprises a body fluid anti-infective composition specifically adapted for incorporation with a standard secondary sponge or wound dressing described previously above, and is intended to be applied for use with this embodiment. In addition, for purposes of this particular application, other sanitary medical/surgical articles including but not limited to wound dressings, swellable sponges, fibrotic packings, and compresses are also fully within the scope of this embodiment.

The body fluid control composition of the preferred embodiment of the present invention comprises a hydrophilic or lipophilic component capable of forming semisolid or solid, ordered or disordered, three dimensional systems or structures upon contact with bodily fluids. Preferably, the hydrophilic or lipophilic component is capable of forming a hydrogel or liquid crystalline system. More preferably, the lipid component defined as being greater than 40% fatty acid, fatty acid monoester, fatty acid diester, or fatty acid triester alone or in combination thereby forming a lipid composition. Most preferably, the lipid composition comprises 15.0%-100% by weight of glyceryl monooleate (GMO), wherein a balance of lipid composition is a polar solvent such as water or a physiologic buffered aqueous system or alternatively a semipolar solvent or a nonpolar solvent. Specific solvents include, but are not limited to alcohol(s), polyethylene glycol(s), propylene glycol, and polypropylene glycol. The lipid composition has a solvent content defined at 0%-90% polar, semi-polar, or nonpolar.

The lipid composition provides a mixture being operable to function as an anti-infective and lubricant within or upon bodily tissues, specifically to facilitate hemorrhoidal and anal fissure treatment, thereby inhibiting the production of and serving to treat infection caused by bacteria, viruses, protozoa, fungi, yeast, and analogous causative agents of infectious diseases independently as a semisolid, gel, or liquid lipid mixture, or as incorporated with a standard secondary sponge or wound dressing.

The lipid composition may incorporate synergistic compound(s) and active medicament(s), wherein the synergistic compound(s) includes but is not limited to physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, hyaluronic acid and hyaluronic acid derivatives, cellulose and cellulose derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes and enzyme inhibitors. The blood products comprise platelets, prothrombin, and fibrinogen.

Alternatively, the standard secondary sponge or wound dressing is envisioned as being incorporated with a fluid control film component to facilitate delivery of active medicaments to bodily tissue.

In order to facilitate delivery of the lipid composition as an independent semisolid, gel, or liquid lipid mixture, the independent lipid composition is delivered or transmitted as a suspension or solution thereof to medical treatment site or effected area via injection, irrigation, stream, or spray.

In order to facilitate delivery of the lipid composition as incorporated with a standard secondary sponge or wound dressing, the lipid composition is applied uniformly to outer surfaces of the sponge or dressing via hot-melt coating method in a manner so as to ensure absorptive capacity of the sponge or dressing is not exceeded. Alternative methods for incorporating the lipid composition to a selected medical article includes general liquid coating by spray, dip, knife spread or cast transfer methodology.

The lipid composition with synergistic and medicament components provides a mixture being operable to function as an anti-infective within or upon bodily tissues, to facilitate treatment of anorectal conditions and pathologies such as hemorrhoids and anal fissures, thereby inhibiting the production of and serving to treat infection caused by bacteria, viruses, protozoa, fungi, yeast, and analogous causative agents of infectious diseases independently as a semisolid, gel, or liquid lipid mixture, or as incorporated with a standard secondary sponge or wound dressing.

It is a further envisionment of the present application that the disclosed system is operable to function as a lubricant system independently as a semisolid, gel or liquid lipid mixture or as incorporated with a standard secondary sponge or wound dressing of other medical or surgical structure to aid in the insertion or removal of said device or structure during or following a medical or surgical treatment. It is envisioned that this system is operable to facilitate the insertion and removal of articles such as packing or sponges following surgical treatment of the mucous membranes of the nose, sinuses or throat.

E. First Alternative, Anti-Infective Dental Product

In order to facilitate dental treatment and dental procedures through optimum moisture balance, an alternative absorbent, anti-infective dental product is disclosed and described hereinbelow. The absorbent, anti-infective, dental product comprises a body fluid anti-infective composition specifically adapted for incorporation with a standard, commercially available dental mouthpiece or tray, or as a component of a dental kit utilized in the dental arts. For purposes of this particular application, other sanitary medical/surgical articles including but not limited to wound dressings, swellable sponges, fibrotic packings, and compresses are also fully within the intended scope of this particular embodiment.

The body fluid control composition of the preferred embodiment of the present invention comprises a hydrophilic or lipophilic component capable of forming semisolid or solid, ordered or disordered, three dimensional systems or structures upon contact with bodily fluids. Preferably, the hydrophilic or lipophilic component is capable of forming a hydrogel or liquid crystalline system. More preferably, the lipid component defined as being greater than 40% fatty acid, fatty acid monoester, fatty acid diester, or fatty acid triester alone or in combination thereby forming a lipid composition. Most preferably, the lipid composition comprises 15.0%-100% by weight of glyceryl monooleate (GMO), wherein a balance of lipid composition is a polar solvent such as water or a physiologic buffered aqueous system or alternatively a semipolar solvent or a nonpolar solvent. Specific solvents include, but are not limited to alcohol(s), polyethylene glycol(s), propylene glycol, and polypropylene glycol. The lipid composition has a solvent content defined at 0%-90% polar, semi-polar, or nonpolar.

The lipid composition is adapted to be utilized independently as a semisolid, gel, or liquid lipid mixture, or as incorporated with a standard dental mouthpiece or tray, or as a component of a dental kit in order to facilitate dental treatment and dental procedures through optimum moisture balance.

The lipid composition may incorporate synergistic compound(s) and active medicament(s), wherein the synergistic compound(s) includes but is not limited to physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, hyaluronic acid and hyaluronic acid derivatives, cellulose and cellulose derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes and enzyme inhibitors, a dental whitener, a gingivitis agent, a periodontal agent, and a fluoride derivative(s). The blood products comprise platelets, prothrombin, and fibrinogen.

In order to facilitate delivery of the lipid composition as an independent semisolid, gel, or liquid lipid mixture, the independent lipid composition is delivered or transmitted as a suspension or solution thereof to medical treatment site or effected area via injection, irrigation, stream, or spray.

In order to facilitate delivery of the lipid composition as incorporated with a standard secondary sponge or wound dressing, the lipid composition is applied uniformly to outer surfaces of the sponge or dressing via hot-melt coating method in a manner so as to ensure absorptive capacity of the sponge or dressing is not exceeded. Alternative methods for incorporating the lipid composition to a selected medical article includes general liquid coating by spray, dip, knife spread or cast transfer methodology.

The lipid composition with synergistic and medicament components provides a mixture being operable to provide for dental treatment and dental procedures through optimum moisture balance either independently as a semisolid, gel, or liquid lipid mixture, or as incorporated with a standard dental mouthpiece or tray, as a component of a dental kit, or as incorporated with a standard secondary sponge or dressing.

A second alternative lipid-based system and combination lipid, multiparticulate system is provided, according to an alternate embodiment of the present invention, comprising an absorbent, anti-infective composition adapted to provide an anti-infective agent for human or veterinary application which inhibits the production of and serves to treat infection caused by bacteria, viruses, protozoa, fungi, yeast, and analogous causative agents of infectious diseases of internal and external wounds or burns resulting from trauma, hemorrhage, injury, disease, or surgical procedures, as well as to treat hemorrhoids and anal fissures before and after surgical intervention, to treat dental wounds, to coat medical device hardware intended for implantation into the body, and to serve as a lubricant for prevention of sexually transmitted diseases. The absorbent, anti-infective composition is adapted to function both independently as a separate lipid mixture, and conjunctively with standard, commercially available medical/surgical articles including, but not limited to bandages, sponges, dressings, prophylactic devices, and related medical products described earlier.

F. Second Alternative Absorbent, Anti-Infective Product

In order to provide an anti-infective agent for internal and external wounds or burns resulting from trauma, hemorrhage, injury, disease, or surgical procedures, a second alternative absorbent, anti-infective product is disclosed and described hereinbelow. The second alternative absorbent, anti-infective product comprises a body fluid anti-infective composition specifically adapted for incorporation with a standard secondary sponge or dressing described above, and is intended to be applied for use with this embodiment. For purposes of this particular application, other sanitary medical articles including but not limited to swellable sponges, fibrotic packings, adhesive bandages, and compresses are also fully within the scope of this embodiment.

The body fluid control composition of the preferred embodiment of the present invention comprises a hydrophilic or lipophilic component capable of forming semisolid or solid, ordered or disordered, three dimensional systems or structures upon contact with bodily fluids. Preferably, the hydrophilic or lipophilic component is capable of forming a hydrogel or liquid crystalline system. More preferably, the lipid component defined as being greater than 40% fatty acid, fatty acid monoester, fatty acid diester, or fatty acid triester alone or in combination thereby forming a lipid composition. Most preferably, the lipid composition comprises 15.0%-100% by weight of glyceryl monooleate (GMO), wherein a balance of lipid composition is a polar solvent such as water or a physiologic buffered aqueous system or alternatively a semipolar solvent or a nonpolar solvent. Specific solvents include, but are not limited to alcohol(s), polyethylene glycol(s), propylene glycol, and polypropylene glycol. The lipid composition has a solvent content defined at 0%-90% polar, semi-polar, or nonpolar.

The lipid composition provides a mixture being operable to function as an anti-infective agent for internal and external wounds or burns independently as a semisolid, gel, or liquid lipid mixture, or as incorporated with a standard secondary sponge or dressing.

The lipid composition, during an initial liquid phase thereof, may incorporate synergistic compound(s) and active medicament(s) as independent granulates, agglomerates or coated particles, rather than being combined as a simple mixture with lipid composition. The synergistic compound(s) includes but is not limited to physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, hyaluronic acid and hyaluronic acid derivatives, cellulose and cellulose derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes and enzyme inhibitors. The blood products comprise platelets, prothrombin, and fibrinogen.

Alternatively, the standard sponge or dressing is envisioned as being incorporated with a fluid control film component to facilitate delivery of active medicaments to bodily tissue.

The granulates, agglomerates, or coated particles are produced by a method known in the art consisting of a fluid bed processes, low shear processes, and high shear processes, precipitation, or coacervation.

The lipid composition with synergistic and medicament components provides a mixture which functions as an anti-infective agent for internal and external wounds or burns independently as a semisolid, gel, or liquid lipid mixture, or as incorporated with a standard secondary sponge or dressing.

In order to facilitate delivery of the lipid composition as an independent semisolid, gel, or liquid lipid mixture, independent lipid composition is delivered or transmitted as a suspension or solution thereof to medical treatment site or effected area via injection, irrigation, stream, or spray.

In order to facilitate delivery of the lipid composition as incorporated with a standard secondary sponge or dressing, the lipid composition is applied uniformly to outer surfaces of the dressing via hot-melt coating method in a manner so as to ensure absorptive capacity of the dressing is not exceeded. Alternative methods for incorporating the lipid composition to a selected medical article includes general liquid coating by spray, dip, knife spread or cast transfer methodology. The resulting product according the aforementioned respective means of transmission is an independent lipid composition and an absorbent, anti-infective secondary sponge or dressing which function as an anti-infective agent for internal and external wounds or burns resulting from trauma, hemorrhage, injury, disease, or surgical procedures.

G. Third Alternative Absorbent, Anti-Infective Product

In order to treat anorectal conditions such as hemorrhoids and anal fissures prior to and following surgical procedures through the introduction of an anti-infective agent, a third alternative absorbent, anti-infective product is disclosed and described hereinbelow. The third alternative absorbent, anti-infective product incorporates a unique combination of elements so as to provide a sanitary medical article being particularly operable to provide an anti-infective agent for treating anorectal conditions. The third alternative absorbent, anti-infective product comprises a body fluid control composition specifically adapted for incorporation with a standard secondary sponge or wound dressing described above, and is intended to be applied for use with this embodiment. For purposes of this particular application, other sanitary medical/surgical articles including but not limited to swellable sponges, fibrotic packings, and compresses are also fully within the scope of this embodiment.

The body fluid control composition of the preferred embodiment of the present invention comprises a hydrophilic or lipophilic component capable of forming semisolid or solid, ordered or disordered, three dimensional systems or structures upon contact with bodily fluids. Preferably, the hydrophilic or lipophilic component is capable of forming a hydrogel or liquid crystalline system. More preferably, the lipid component defined as being greater than 40% fatty acid, fatty acid monoester, fatty acid diester, or fatty acid triester alone or in combination thereby forming a lipid composition. Most preferably, the lipid composition comprises 15.0%-100% by weight of glyceryl monooleate (GMO), wherein a balance of lipid composition is a polar solvent such as water or a physiologic buffered aqueous system or alternatively a semipolar solvent or a nonpolar solvent. Specific solvents include, but are not limited to alcohol(s), polyethylene glycol(s), propylene glycol, and polypropylene glycol. The lipid composition has a solvent content defined at 0%-90% polar, semi-polar, or nonpolar.

The lipid composition provides a mixture being operable for utilization as an anti-infective agent for treating hemorrhoids and anal fissures prior to and following surgical intervention independently as a semisolid, gel, or liquid lipid mixture, or as incorporated with a standard secondary sponge or wound dressing.

The lipid composition may incorporate synergistic compound(s) and active medicament(s). The synergistic compound(s) includes but is not limited to physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, hyaluronic acid and hyaluronic acid derivatives, cellulose and cellulose derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes and enzyme inhibitors. The blood products comprise platelets, prothrombin, and fibrinogen.

Alternatively, the standard secondary sponge or wound dressing is envisioned as being incorporated with a fluid control film component to facilitate delivery of active medicaments to bodily tissue.

In order to facilitate delivery of the lipid composition as an independent semisolid, gel, or liquid lipid mixture, the independent lipid composition is delivered or transmitted as a suspension or solution thereof to medical treatment site or effected area via injection, irrigation, stream, or spray.

The lipid composition with synergistic and medicament components provides a mixture being operable for utilization as an anti-infective agent for treating anorectal conditions such as hemorrhoids and anal fissures prior to and following surgical intervention independently as a semisolid, gel, or liquid lipid mixture, or as incorporated with a standard secondary sponge or wound dressing.

In order to facilitate delivery of the lipid composition as incorporated with a standard secondary sponge or wound dressing, the lipid composition is applied uniformly to outer surfaces of the sponge or dressing via hot-melt coating method in a manner so as to ensure absorptive capacity of the sponge or dressing is not exceeded. Alternative methods for incorporating the lipid composition to a selected medical article includes general liquid coating by spray, dip, knife spread or cast transfer methodology. The resulting product according to both respective means of transmission is an independent lipid composition and an absorbent, anti-infective secondary sponge or dressing which function to provide an anti-infective agent for treating anorectal conditions such as hemorrhoids and anal fissures prior to and following surgical intervention relative thereto, thereby inhibiting the production of and serving to treat infection caused by bacteria, viruses, protozoa, fungi, yeast, and analogous causative agents of infectious diseases.

H. Second Alternative, Anti-Infective Dental Product

In order to facilitate dental wound treatment resulting from trauma, hemorrhage, injury, disease, or surgical/dental procedures through the application of an anti-infective agent, a second alternative, anti-infective dental product is disclosed and described hereinbelow. The second alternative, anti-infective, dental product comprises a body fluid anti-infective composition specifically adapted for incorporation with a standard dental mouthpiece or tray, or as a component of a dental kit utilized in the dental arts. For purposes of this particular application, other sanitary medical/surgical articles including but not limited to wound dressings, swellable sponges, fibrotic packings, and compresses are also fully within the intended scope of this particular embodiment.

The body fluid control composition of the preferred embodiment of the present invention comprises a hydrophilic or lipophilic component capable of forming semisolid or solid, ordered or disordered, three dimensional systems or structures upon contact with bodily fluids. Preferably, the hydrophilic or lipophilic component is capable of forming a hydrogel or liquid crystalline system. More preferably, the lipid component defined as being greater than 40% fatty acid, fatty acid monoester, fatty acid diester, or fatty acid triester alone or in combination thereby forming a lipid composition. Most preferably, the lipid composition comprises 15.0%-100% by weight of glyceryl monooleate (GMO), wherein a balance of lipid composition is a polar solvent such as water or a physiologic buffered aqueous system or alternatively a semipolar solvent or a nonpolar solvent. Specific solvents include, but are not limited to alcohol(s), polyethylene glycol(s), propylene glycol, and polypropylene glycol. The lipid composition has a solvent content defined at 0%-90% polar, semi-polar, or nonpolar.

The lipid composition is adapted to be utilized independently as a semisolid, gel, or liquid lipid mixture, or as incorporated with a standard dental mouthpiece or tray, or as a component of a dental kit in order to facilitate dental wound treatment through the application of an anti-infective agent.

The lipid composition may incorporate synergistic compound(s) and active medicament(s), wherein the synergistic compound(s) includes but is not limited to physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, hyaluronic acid and hyaluronic acid derivatives, cellulose and cellulose derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes and enzyme inhibitors a gingivitis agent, or a periodontal agent. The blood products comprise platelets, prothrombin, and fibrinogen.

In order to facilitate delivery of the lipid composition as an independent semisolid, gel, or liquid lipid mixture, the independent lipid composition is delivered or transmitted as a suspension or solution thereof to medical treatment site or effected area via injection, irrigation, stream, or spray.

In order to facilitate delivery of the lipid composition as incorporated with a standard dental mouthpiece or tray, secondary sponge or wound dressing, the lipid composition is applied uniformly to outer surfaces of the tray, sponge or dressing via hot-melt coating method in a manner so as to ensure absorptive capacity of the article is not exceeded. Alternative methods for incorporating the lipid composition to a selected medical article includes general liquid coating by spray, dip, knife spread or cast transfer methodology.

The lipid composition with synergistic and medicament components provides a mixture being operable to provide dental wound and burn treatment through the application of an anti-infective agent either independently as a semisolid, gel, or liquid lipid mixture, or as incorporated with a standard dental mouthpiece or tray, as a component of a dental kit, or as incorporated with a standard secondary sponge or dressing.

I. Medical Device Hardware Coating

A medical device hardware coating is disclosed which functions to provide standard medical device hardware with an anti-infective coating. The coating is envisioned for use with standard medical hardware particularly adapted for implantation into the body. The medical device hardware coating comprises an anti-infective coating composition specifically adapted for independent use as a semisolid, gel, or liquid lipid mixture, or as incorporated with, or as a component of a prior or subsequent coating or film. For purposes of this particular application, medical device hardware includes but is not limited to joint or partial limb prosthetics, dental devices and prosthetics, stents, shunts, and related dental and orthopedic hardware.

The body fluid control composition of the preferred embodiment of the present invention comprises a hydrophilic or lipophilic component capable of forming semisolid or solid, ordered or disordered, three dimensional systems or structures upon contact with bodily fluids. Preferably, the hydrophilic or lipophilic component is capable of forming a hydrogel or liquid crystalline system. More preferably, the lipid component defined as being greater than 40% fatty acid, fatty acid monoester, fatty acid diester, or fatty acid triester alone or in combination thereby forming a lipid composition. Most preferably, the lipid composition comprises 15.0%-100% by weight of glyceryl monooleate (GMO), wherein a balance of lipid composition is a polar solvent such as water or a physiologic buffered aqueous system or alternatively a semipolar solvent or a nonpolar solvent. Specific solvents include, but are not limited to alcohol(s), polyethylene glycol(s), propylene glycol, and polypropylene glycol. The lipid composition has a solvent content defined at 0%-90% polar, semi-polar, or nonpolar.

The lipid composition is adapted to be utilized independently as a semisolid, gel, or liquid lipid mixture, or as incorporated with standard, commercially available orthopedic/dental hardware adapted for implantation into the body.

The lipid composition may incorporate synergistic compound(s) and active medicament(s), wherein the synergistic compound(s) includes but is not limited to physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, hyaluronic acid and hyaluronic acid derivatives, cellulose and cellulose derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes and enzyme inhibitors. The blood products comprise platelets, prothrombin, and fibrinogen.

In order to facilitate delivery of the lipid composition as an independent semisolid, gel, or liquid lipid mixture, the independent lipid composition is delivered or transmitted as a suspension or solution thereof to standard medical device hardware or medical treatment site via injection, irrigation, stream, or spray.

Additional methods for incorporating the lipid composition to a selected medical article includes general liquid coating by spray, dip, knife spread or cast transfer methodology. Other suitable methods for effectuating delivery of the lipid composition either independently or as incorporated with, or as a component of a prior or subsequent coating or film is further envisioned. Furthermore, obvious medical hardware coating modifications which allow the invention's principles to apply to unpublished current medical hardware coating delivery methods and future industry standards are also considered within invention's scope.

The lipid composition with synergistic and medicament components functions to provide an anti-infective coating composition either independently as a semisolid, gel, or liquid lipid mixture, or as incorporated with standard medical device hardware to serve as an anti-infective coating therefore.

J. Sexually Transmitted Disease Prevention Lubricant

A lubricant adapted for the prevention of sexually transmitted diseases is disclosed. The lubricant comprises an anti-infective composition specifically adapted for independent use as a semisolid, gel, or liquid lipid mixture, or as incorporated with, or as a component of a standard birth control or prophylactic device. For purposes of this particular application, a birth control or prophylactic device includes but is not limited to condoms, diaphragms, and related contraceptive barrier devices and products.

The body fluid control composition of the preferred embodiment of the present invention comprises a hydrophilic or lipophilic component capable of forming semisolid or solid, ordered or disordered, three dimensional systems or structures upon contact with bodily fluids. Preferably, the hydrophilic or lipophilic component is capable of forming a hydrogel or liquid crystalline system. More preferably, the lipid component defined as being greater than 40% fatty acid, fatty acid monoester, fatty acid diester, or fatty acid triester alone or in combination thereby forming a lipid composition. Most preferably, the lipid composition comprises 15.0%-100% by weight of glyceryl monooleate (GMO), wherein a balance of lipid composition is a polar solvent such as water or a physiologic buffered aqueous system or alternatively a semipolar solvent or a nonpolar solvent. Specific solvents include, but are not limited to alcohol(s), polyethylene glycol(s), propylene glycol, and polypropylene glycol. The lipid composition has a solvent content defined at 0%-90% polar, semi-polar, or nonpolar.

The lipid composition is adapted to be utilized independently as a semisolid, gel, or liquid lipid mixture, or as incorporated with standard birth control or prophylactic device.

The lipid composition may incorporate synergistic compound(s) and active medicament(s), wherein the synergistic compound(s) includes but is not limited to physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, hyaluronic acid and hyaluronic acid derivatives, cellulose and cellulose derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes and enzyme inhibitors. The blood products comprise platelets, prothrombin, and fibrinogen.

In order to facilitate delivery of the lipid composition as an independent semisolid, gel, or liquid lipid mixture, the independent lipid composition is delivered or transmitted as a suspension or solution thereof to the effected area or medical treatment site via vaginal plunger, injection, irrigation, stream, or spray.

Alternatively, in order to facilitate delivery of the lipid composition as incorporated with a standard birth control or prophylactic device, the lipid composition is applied uniformly to surfaces of the prophylactic device via hot-melt coating method, or via general liquid coating by spray, dip, knife spread or cast transfer methodology. Other suitable methods for effectuating delivery of the lipid composition either independently or as incorporated with, or as a component of a birth control or prophylactic device is further envisioned. In addition, obvious birth control or prophylactic device coating modifications which allow the invention's principles to apply to unpublished current prophylactic coating delivery methods and future industry standards are also considered within invention's scope.

The lipid composition with synergistic and medicament components provides a mixture facilitating the prevention of sexually transmitted diseases either independently as a semisolid, gel, or liquid lipid mixture, or as incorporated with a standard birth control or prophylactic device.

K. Thrombin-Inhibitory, Hemostatic Tissue Sealant as a Neuroprotective System for Normal Neural Tissue

In order to facilitate exudate absorption and hemostasis within the central nervous system resulting from trauma, hemorrhage, injury, disease or surgical procedures, a hemostatic tissue sealant with thrombin-inhibitory properties is disclosed and described hereinbelow. The system disclosed possesses the inherent ability to inhibit the formation of thrombin, a factor of the clotting cascade. In addition to the normal function of thrombin in facilitating the coagulation of blood, it exhibits a concentration dependant local toxicity in certain tissues of the body such as neural tissue of the central nervous system. The inhibition of the formation of thrombin therefore reduces or eliminates the local toxicity and inflammation thereby preserving the viability and function of the neural tissue. The thrombin-inhibitory hemostatic tissue sealant incorporates a unique combination of elements so as to provide both an independent lipid mixture and a sanitary medical article being particularly operable to facilitate exudate absorption and hemostasis for spontaneous and surgical bleeding within the central nervous system. The thrombin-inhibitory hemostatic tissue sealant comprises a body fluid control composition adapted for independent use or incorporation with a surgical sponge or wound dressing. For purposes of this particular application, other sanitary medical articles including but not limited to swellable sponges, fibrotic packings, and compresses are also fully within the scope of this embodiment.

The thrombin-inhibitory hemostatic tissue sealant composition comprises a hydrophilic or lipophilic component capable of forming semisolid or solid, ordered or disordered, three dimensional systems or structures upon contact with bodily fluids. Preferably, the hydrophilic or lipophilic component is capable of forming a hydrogel or liquid crystalline system. More preferably, the lipid component defined as being greater than 40% fatty acid, fatty acid monoester, fatty acid diester, or fatty acid triester alone or in combination thereby forming a lipid composition. Most preferably, the lipid composition comprises 15.0%-100% by weight of glyceryl monooleate (GMO), wherein a balance of lipid composition is a polar solvent such as water or a physiologic buffered aqueous system or alternatively a semipolar solvent or a nonpolar solvent. Specific solvents include, but are not limited to alcohol(s), polyethylene glycol(s), propylene glycol, and polypropylene glycol. The lipid composition has a solvent content defined at 0%-90% polar, semi-polar, or nonpolar.

The thrombin-inhibitory hemostatic tissue sealant composition provides a mixture being operable to facilitate absorption of body fluids and hemostasis for central nervous system bleeding independently as a semisolid, gel, or liquid lipid mixture, or as incorporated with a secondary sponge or dressing.

The thrombin-inhibitory hemostatic tissue sealant composition, during an initial liquid phase thereof, may incorporate synergistic compound(s) and active medicament(s) as independent granulates, agglomerates or coated particles, rather than being combined as a simple mixture with lipid composition. The synergistic compound(s) includes physiologically compatible monovalent, divalent and trivalent ions and salts thereof, calcium derivatives, potassium derivatives, sulfate derivatives, phospholipids, fatty acids, gelatin and gelatin derivatives, hyaluronic acid and hyaluronic acid derivatives, cellulose and cellulose derivatives, collagen, chitosan derivatives, hydrogel forming agents, hydrocolloid forming agents, alginic acid derivatives, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes and enzyme inhibitors, said synergistic compounds being operable to facilitate absorption of body fluids, complete or partial clotting of blood, bloody discharge or exudates upon contact. The blood products comprise platelets, prothrombin, and fibrinogen.

The granulates, agglomerates, or coated particles are produced by a methods known in the art such as fluid bed processes, a low shear processes, high shear processes, precipitation and coacervation as non-limiting examples.

The thrombin-inhibitory hemostatic tissue sealant composition with synergistic and medicament components provides a mixture being operable to facilitate absorption of body fluids and hemostasis for central nervous system bleeding independently as a semisolid, gel, or liquid lipid mixture, or as incorporated with a surgical dressing, sponge or other absorbent article.

In order to facilitate delivery of the thrombin-inhibitory hemostatic tissue sealant composition as an independent semisolid, gel, or liquid lipid mixture, independent lipid composition is delivered or transmitted as a suspension or solution thereof to medical treatment site or effected area via injection, irrigation, stream, or spray.

In order to facilitate delivery of the thrombin-inhibitory hemostatic tissue sealant composition as incorporated with a dressing, sponge, or other absorbent article, the lipid composition is applied uniformly to outer surfaces of the dressing via hot-melt coating method in a manner so as to ensure absorptive capacity of the dressing is not exceeded. Alternative methods for incorporating the lipid composition to a selected medical article includes general liquid coating by spray, dip, knife spread or cast transfer methodology. The resulting product according the aforementioned respective means of transmission is an independent lipid composition and an exudate-absorbent, hemostatic secondary sponge or dressing which function to facilitate exudate absorption and hemostasis for central nervous system bleeding resulting from trauma, hemorrhage, injury, disease, or surgical procedures.

L. Thrombin-Inhibitory Drug Delivery System for Adjunct Therapy for Malignant Tumors

A thrombin-inhibitory drug delivery system for adjunct therapy for malignant tumors is disclosed and described hereinbelow. The system disclosed possesses the inherent ability to inhibit the formation of thrombin, a factor of the clotting cascade. In addition to the normal function of thrombin in facilitating the coagulation of blood, it exhibits potentially important effects in metastatic, proliferative and angiogenic functions particularly in malignant tumors such as those of the central nervous system. The inhibition of the formation of thrombin therefore reduces or eliminates the effects of thrombin thereby reducing the viability and proliferative ability of malignant tumors. The thrombin-inhibitory drug delivery system incorporates a unique combination of elements so as to provide both an independent lipid mixture and lipid mixture containing multiparticulates being particularly operable to facilitate drug delivery of a wide variety of chemotherapeutic agents alone or in combination and inhibit malignant tumor growth within the central nervous system.

The thrombin-inhibitory drug delivery composition comprises a hydrophilic or lipophilic component capable of forming semisolid or solid, ordered or disordered, three dimensional systems or structures upon contact with bodily fluids. Preferably, the hydrophilic or lipophilic component is capable of forming a hydrogel or liquid crystalline system. More preferably, the lipid component defined as being greater than 40% fatty acid, fatty acid monoester, fatty acid diester, or fatty acid triester alone or in combination thereby forming a lipid composition. Most preferably, the lipid composition comprises 15.0%-100% by weight of glyceryl monooleate (GMO), wherein a balance of lipid composition is a polar solvent such as water or a physiologic buffered aqueous system or alternatively a semipolar solvent or a nonpolar solvent. Specific solvents include, but are not limited to alcohol(s), polyethylene glycol(s), propylene glycol, and polypropylene glycol. The lipid composition has a solvent content defined at 0%-90% polar, semi-polar, or nonpolar.

The thrombin-inhibitory drug delivery composition provides a mixture being operable to facilitate inhibition of thrombin induced metastatic, proliferative, and angiogenic actions and further serve to deliver single or multiple chemotherapeutic agents for synergistic therapy of malignant tumors particularly of the central nervous system.

The thrombin-inhibitory drug delivery composition, during an initial liquid phase thereof, may incorporate synergistic compound(s) and active medicament(s) as independent granulates, agglomerates or coated particles, rather than being combined as a simple mixture with lipid composition. The synergistic compound(s) includes physiologically compatible monovalent, divalent and trivalent ions and salts thereof, calcium derivatives, potassium derivatives, sulfate derivatives, phospholipids, fatty acids, gelatin and gelatin derivatives, hyaluronic acid and hyaluronic acid derivatives, cellulose and cellulose derivatives, collagen, chitosan derivatives, hydrogel forming agents, hydrocolloid forming agents, alginic acid derivatives, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes, enzyme inhibitors, and cytotoxic agents.

The granulates, agglomerates, or coated particles are produced by a methods known in the art such as fluid bed processes, a low shear processes, high shear processes, precipitation and coacervation as non-limiting examples.

In order to facilitate delivery of the thrombin-inhibitory drug delivery composition as an independent semisolid, gel, or liquid lipid mixture, independent lipid composition is delivered or transmitted as a suspension or solution thereof to medical treatment site or effected area via injection, irrigation, or implantation.

M. Vascular Plug

A system utilized as a hemostatic vascular plug or patch for arterial or large venous vessels is disclosed and described hereinbelow. The system disclosed possesses an inherent hemostatic quality and further possesses the ability to resist removal or displacement by arterial blood pressures. The vascular plug system incorporates a unique combination of elements so as to provide both an independent lipid mixture and lipid mixture containing multiparticulates being particularly operable to establish hemostasis required at the site of an arterial puncture or arterial line placement for medical testing or therapy.

The vascular plug composition comprises a hydrophilic or lipophilic component capable of forming semisolid or solid, ordered or disordered, three dimensional systems or structures upon contact with bodily fluids. Preferably, the hydrophilic or lipophilic component is capable of forming a hydrogel or liquid crystalline system. More preferably, the lipid component defined as being greater than 40% fatty acid, fatty acid monoester, fatty acid diester, or fatty acid triester alone or in combination thereby forming a lipid composition. Most preferably, the lipid composition comprises 15.0%-100% by weight of glyceryl monooleate (GMO), wherein a balance of lipid composition is a polar solvent such as water or a physiologic buffered aqueous system or alternatively a semipolar solvent or a nonpolar solvent. Specific solvents include, but are not limited to alcohol(s), polyethylene glycol(s), propylene glycol, and polypropylene glycol. The lipid composition has a solvent content defined at 0%-90% polar, semi-polar, or nonpolar.

The vascular plug composition provides a mixture being operable to facilitate the hemostasis at the site of a vascular or arterial puncture or breach as would be common in the medical placement of indwelling arterial or central venous catheters for diagnostic and medical treatment.

The vascular plug composition, during an initial liquid phase thereof, may incorporate synergistic compound(s) and active medicament(s) as independent granulates, agglomerates or coated particles, rather than being combined as a simple mixture with lipid composition. The synergistic compound(s) includes physiologically compatible monovalent, divalent and trivalent ions and salts thereof, calcium derivatives, potassium derivatives, sulfate derivatives, phospholipids, fatty acids, gelatin and gelatin derivatives, hyaluronic acid and hyaluronic acid derivatives, cellulose and cellulose derivatives, collagen, chitosan derivatives, hydrogel forming agents, hydrocolloid forming agents, alginic acid derivatives, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes, and enzyme inhibitors.

The granulates, agglomerates, or coated particles are produced by a methods known in the art such as fluid bed processes, a low shear processes, high shear processes, precipitation and coacervation as non-limiting examples.

In order to facilitate delivery of the vascular plug composition as an independent semisolid, gel, or liquid lipid mixture, independent lipid composition is delivered or transmitted as a suspension or solution thereof to medical treatment site or effected area via injection, irrigation, or implantation.

N. Cosmetic Augmentation

A system utilized for cosmetic tissue augmentation is disclosed and described hereinbelow. The system disclosed possesses an inherent biocompatible quality that allows direct injection or implantation into tissues for the purpose of enhancing the aesthetic appearance. The cosmetic augmentation system incorporates a unique combination of elements so as to provide both an independent lipid mixture being particularly operable for injection or implantation into tissues as an independent mixture or as a fill media for an implantable augmentation device.

The cosmetic augmentation composition comprises a hydrophilic or lipophilic component capable of forming semisolid or solid, ordered or disordered, three dimensional systems or structures upon contact with bodily fluids. Preferably, the hydrophilic or lipophilic component is capable of forming a hydrogel or liquid crystalline system. More preferably, the lipid component defined as being greater than 40% fatty acid, fatty acid monoester, fatty acid diester, or fatty acid triester alone or in combination thereby forming a lipid composition. Most preferably, the lipid composition comprises 15.0%-100% by weight of glyceryl monooleate (GMO), wherein a balance of lipid composition is a polar solvent such as water or a physiologic buffered aqueous system or alternatively a semipolar solvent or a nonpolar solvent. Specific solvents include, but are not limited to alcohol(s), polyethylene glycol(s), propylene glycol, and polypropylene glycol. The lipid composition has a solvent content defined at 0%-90% polar, semi-polar, or nonpolar.

The cosmetic augmentation composition provides a mixture being operable for injection or implantation into tissues to facilitate increased volume within the tissues to provide aesthetic augmentation.

The cosmetic augmentation composition, during an initial liquid phase thereof, may incorporate synergistic compound(s) and active medicament(s) as independent granulates, agglomerates or coated particles, rather than being combined as a simple mixture with lipid composition. The synergistic compound(s) includes physiologically compatible monovalent, divalent and trivalent ions and salts thereof, calcium derivatives, potassium derivatives, sulfate derivatives, phospholipids, fatty acids, gelatin and gelatin derivatives, hyaluronic acid and hyaluronic acid derivatives, hyaluronic acid and hyaluronic acid derivatives, cellulose and cellulose derivatives, collagen, chitosan derivatives, hydrogel forming agents, hydrocolloid forming agents, alginic acid derivatives, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes, and enzyme inhibitors.

The granulates, agglomerates, or coated particles are produced by a methods known in the art such as fluid bed processes, a low shear processes, high shear processes, precipitation and coacervation as non-limiting examples.

In order to facilitate delivery of the cosmetic augmentation composition as an independent semisolid, gel, or liquid lipid mixture, independent lipid composition is delivered or transmitted as a suspension or solution thereof to medical treatment site or effected area via injection or implantation.

### 3. Operation of the Preferred Embodiment

To use the present invention, user delivers the lipid or lipid combination as an independent composition to the medical treatment site or effected area via injection, irrigation, stream, or spray. Optionally, the lipid or lipid combination is incorporated with a standard, commercially available catamenial absorption product, medical or surgical absorbent article and utilized in the manner commonly practiced and employed when using such product.

Therefore, the foregoing description is included to illustrate the operation of the preferred embodiment and is not meant to limit the scope of the invention. As one can envision, an individual skilled in the relevant art, in conjunction with the present teachings, would be capable of incorporating many minor modifications that are anticipated within this disclosure. The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents. Therefore, the scope of the invention is to be broadly limited only by the following Claims.

What is claimed is:

1. A semisolid system and combination semisolid, multiparticulate system for controlling biological fluids and providing local effects comprising:
a body fluid control composition that will form or maintain a semisolid secondary structure in an aqueous or polar solvent environment, said body fluid control composition being adapted for utilization both as an independent mixture for direct application upon or within body tissues for human or veterinary applications via physical transfer, irrigation, douche, stream, enema or spray and for incorporation with a standard, commercially available medical products in order to facilitate absorption of body fluids, provide local inherent effects or provide hemostasis.

2. The semisolid system and combination semisolid, multiparticulate system for controlling biological fluids and providing local effects of claim 1, wherein said body fluid control composition comprises a liquid crystal-based system.

3. The semisolid system and combination semisolid, multiparticulate system for controlling biological fluids and providing local effects of claim 1, wherein said semisolid system comprises a greater than 40% fatty acid, fatty acid monoester, fatty acid diester, or fatty acid triester alone or in combination and no less than one of said fatty acid includes at least one unsaturated carbon atom, thereby forming a lipid composition.

4. The semisolid system and combination semisolid, multiparticulate system for controlling biological fluids and providing local effects of claim 3, wherein said semisolid composition comprises 25.0% to 100% by weight of glyceryl monooleate, wherein a balance of said semisolid composition is a solvent, wherein said semisolid composition having a solvent content defined at 0% to 90% polar, 0% to 90% semi-polar, or 0% to 90% nonpolar, thereby providing a mixture being operable to facilitate absorption of body fluids, partial clotting of menstrual or otherwise bloody discharge upon contact within, upon or in close proximity to female genitalia as an independent semisolid, gel, or liquid mixture.

5. The semisolid system and combination semisolid, multiparticulate system for controlling biological fluids and providing local effects of claim 1, 2, 3 or 4, wherein said semisolid composition further comprises synergistic compounds or active medicaments used to alter or otherwise strengthen the semisolid structure matrix or consistency, or enhance the physiologic function of the system alone or in combination.

6. The semisolid system and combination semisolid, multiparticulate system for controlling biological fluids and providing local effects of claim 5, wherein said synergistic compounds include physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, cellulose and cellulose derivatives, hyaluronic and hyaluronic derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes and enzyme inhibitors, said synergistic compounds being operable to facilitate absorption of body fluids, complete or partial clotting of blood, bloody discharge or exudates upon contact within, upon or in close proximity to female genitalia as an independent semisolid, gel, or liquid mixture.

7. The semisolid system and combination semisolid, multiparticulate system for controlling biological fluids and providing local effects of claim 1, wherein said solvent includes alcohol(s), polyethylene glycol(s), propylene glycol, polypropylene glycol, water, and an isotonic aqueous system or a physiologic buffered aqueous system.

8. The semisolid system and combination semisolid, multiparticulate system for controlling biological fluids and providing local effects of claim 6, wherein said oleaginous ointment base(s) comprises petrolatum, white petrolatum, waxes, cetyl esters wax, oleic acid, olive oil, and spermaceti as nonlimiting examples.

9. The semisolid system and combination semisolid, multiparticulate system for controlling biological fluids and providing local effects of claim 6, wherein said absorbent ointment base(s) comprise lanolin, hydrophilic petrolatum, hydroxystearin sulfate or components thereof as nonlimiting examples.

10. The semisolid system and combination semisolid, multiparticulate system for controlling biological fluids and providing local effects of claim 6, wherein said emulsion ointment base(s) comprise cetyl alcohol, stearic acid, hydrophilic ointment, cold cream or components thereof as nonlimiting examples.

11. The semisolid system and combination semisolid, multiparticulate system for controlling biological fluids and providing local effects as in claims 1, 2, 3, 4 or 5, wherein said semisolid composition is incorporated with said standard, commercially available medical product, thereby facilitating absorption of body fluids, partial clotting of menstrual or otherwise bloody discharge upon contact within, upon or in close proximity to female genitalia.

12. The semisolid system and combination semisolid, multiparticulate system for controlling biological fluids and providing local effects of claim 11 wherein said standard, commercially available medical product is a standard, commercially available catamenial absorption article thereby providing a self lubricated exudate-absorbent article operable for facilitating absorption of body fluids, partial clotting of menstrual or otherwise bloody discharge upon contact within, upon or in close proximity to female genitalia.

13. The semisolid system and combination semisolid, multiparticulate system for controlling biological fluids and providing local effects of claim 12, wherein said catamenial absorption article is defined as including a standard, commercially available feminine tampon, menstrual pad, and feminine napkin.

14. The semisolid system and combination semisolid, multiparticulate system for controlling biological fluids and providing local effects as in any of claim 5, wherein said synergistic compounds include physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, cellulose and cellulose derivatives, hyaluronic and hyaluronic derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes and enzyme inhibitors, said synergistic compounds being operable to facilitate absorption of body fluids, partial clotting of menstrual or otherwise bloody discharge or exudates upon contact within, upon or in close proximity to female genitalia as an independent semisolid, gel, or liquid mixture.

15. The semisolid system and combination semisolid, multiparticulate system for controlling biological fluids and providing local effects of claim 14, wherein said blood products includes but is not limited to platelets, prothrombin, thrombin, fibrin, fibrinogen, thromboplastin, and all clotting factors.

16. The semisolid system and combination semisolid, multiparticulate system for controlling biological fluids and providing local effects as in any of claims 5, 14, or 15, wherein said semisolid composition is adapted to be particularly operable to facilitate absorption of body fluids, hemostasis for postpartum, post-operative or general feminine reproductive tract bleeding as an independent semisolid, gel, or liquid mixture.

17. The semisolid system and combination semisolid, multiparticulate system for controlling biological fluids and providing local effects of claim 16, wherein said semisolid composition is adapted to be incorporated with a standard, commercially available catamenial absorption article, douche, irrigation or a standard, commercially available sanitary medical absorbent article.

18. The semisolid system and combination semisolid, multiparticulate system for controlling biological fluids and providing local effects of claim 17, wherein said catamenial absorption article includes but is not limited to a feminine tampon, a menstrual pad, and feminine napkin, thereby providing a sanitary, absorbent feminine article particularly adapted to facilitate absorption of body fluids, hemostasis for postpartum, post-operative hemorrhaging or general feminine reproductive tract bleeding.

19. The semisolid system and combination semisolid, multiparticulate system for controlling biological fluids and providing local effects of claim 17, wherein said sanitary medical absorbent article includes but is not limited to a secondary sponge, a wound dressing, a swellable sponge, a fibrotic packing, a wound packing, and a compress, thereby providing a sanitary, medical absorbent article particularly adapted to facilitate absorption of body fluids, hemostasis for postpartum, post-operative hemorrhaging or general feminine reproductive tract bleeding.

20. The semisolid system and combination semisolid, multiparticulate system for controlling biological fluids and providing local effects of claim 3, wherein said semisolid composition comprises 25.0% to 100% by weight of glyceryl monooleate, wherein a balance of said semisolid composition is a solvent, wherein said semisolid composition having a solvent content defined at 0% to 90% polar, 0% to 90% semi-polar, or 0% to 90% nonpolar, thereby providing an independent semisolid, gel, or liquid mixture adapted to facilitate exudate absorption and hemostasis for internal and external bleeding resulting from trauma, hemorrhage, injury, disease, or surgical procedures in or upon any tissue without limitation.

21. The semisolid system and combination semisolid, multiparticulate system for controlling biological fluids and providing local effects of claim 20, wherein said semisolid composition further comprises synergistic compounds or active medicaments used to alter or otherwise strengthen the semisolid structure matrix or consistency, or enhance the physiologic function of the system alone or in combination.

22. The semisolid system and combination semisolid, multiparticulate system for controlling biological fluids and providing local effects of claim 21, wherein said synergistic compounds include physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, cellulose and cellulose derivatives, hyaluronic and hyaluronic derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes and enzyme inhibitors, said synergistic compounds being operable to facilitate exudate absorption and hemostasis for internal and external bleeding resulting from trauma, hemorrhage, injury, disease, or surgical procedures in or upon any tissue without limitation as an independent semisolid, gel, or liquid mixture.

23. The semisolid system and combination semisolid, multiparticulate system for controlling biological fluids and providing local effects of claim 22, wherein said blood products includes but is not limited to platelets, prothrombin, thrombin, fibrin, fibrinogen, thromboplastin, and all clotting factors.

24. The semisolid system and combination semisolid, multiparticulate system for controlling biological fluids and providing local effects of claim 1, 2, 3, 20, or 21, wherein said semisolid composition is adapted to be incorporated with a standard, commercially available sanitary medical absorbent article.

25. The semisolid system and combination semisolid, multiparticulate system for controlling biological fluids and providing local effects of claim 24, wherein said sanitary medical absorbent article includes but is not limited to a secondary sponge, a wound dressing, a swellable sponge, a fibrotic packing, a wound packing, an adhesive bandage, and a compress, thereby providing a self lubricated, exudate-absorbent, hemostatic tissue sealant article being particularly operable to facilitate exudate absorption and hemostasis for internal and external bleeding resulting from trauma, hemorrhage, injury, disease, or surgical procedures in or upon any tissue without limitation.

26. The semisolid system and combination semisolid, multiparticulate system for controlling biological fluids and providing local effects of claim 3, wherein said semisolid composition comprises 25.0% to 100% by weight of glyceryl monooleate, wherein a balance of said semisolid composition is a solvent, wherein said semisolid composition having a solvent content defined at 0% to 90% polar, 0% to 90% semi-polar, or 0% to 90% nonpolar, thereby providing an independent semisolid, gel, or liquid mixture adapted to facilitate exudate absorption and hemostasis for internal and external dental or gingival bleeding originating within or near oropharangeal region including the mouth, teeth, gums or throat resulting from trauma, hemorrhage, injury, disease, or dental procedures.

27. The semisolid system and combination semisolid, multiparticulate system for controlling biological fluids and providing local effects of claims 1, wherein said semisolid composition further comprises synergistic compounds or active medicaments used to alter or otherwise strengthen the semisolid structure matrix or consistency, or enhance the physiologic function of the system alone or in combination, thereby adapted to facilitate exudate absorption and hemostasis for internal and external dental or gingival bleeding.

28. The semisolid system and combination semisolid, multiparticulate system for controlling biological fluids and providing local effects of claim 27, wherein said synergistic compounds include physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, cellulose and cellulose derivatives, hyaluronic and hyaluronic derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes and enzyme inhibitors, gingivitis agents, periodontal agents, and fluoride derivatives, said synergistic compounds being operable to facilitate absorption of body fluids, partial or complete clotting of blood, bloody discharge or exudates as an independent semisolid, gel, or liquid mixture.

29. The semisolid system and combination semisolid, multiparticulate system for controlling biological fluids and providing local effects of claim 28, wherein said blood products includes but is not limited to platelets, prothrombin, thrombin, fibrin, fibrinogen, thromboplastin, and all clotting factors.

30. The semisolid system and combination semisolid, multiparticulate system for controlling biological fluids and providing local effects of claim 1, 2, 3, 26, 27 or 28, wherein said semisolid composition is adapted to be incorporated with a standard, commercially available sanitary medical absorbent article or part of a dental kit.

31. The semisolid system and combination semisolid, multiparticulate system for controlling biological fluids and providing local effects of claim 30, wherein said sanitary medical absorbent article includes but is not limited to a secondary sponge, a wound dressing, a swellable sponge, a wound packing, and a fibrotic packing, thereby providing an exudate-absorbent, hemostatic tissue sealant article being particularly operable to facilitate exudate absorption and hemostasis for internal and external dental or gingival bleeding resulting from trauma, hemorrhage, injury, disease or dental procedures.

32. The semisolid system and combination semisolid, multiparticulate system for controlling biological fluids and providing local effects as in claim 3, wherein said semisolid composition comprises 25.0% to 100% by weight of glyceryl monooleate, wherein a balance of said semisolid composition is a solvent, wherein said semisolid composition having a solvent content defined at 0% to 90% polar, 0% to 90% semi-polar, or 0% to 90% nonpolar, thereby providing an independent semisolid, gel, or liquid mixture adapted to facilitate exudate absorption and hemostasis for internal and external bleeding of the gastrointestinal tract including hemorrhoids and anal fissures resulting from trauma, hemorrhage, injury, disease or surgical procedures.

33. The semisolid system and combination semisolid, multiparticulate system for controlling biological fluids and providing local effects of claim 1, wherein said semisolid composition further comprises synergistic compounds or active medicaments used to alter or otherwise strengthen the semisolid structure matrix or consistency, or enhance the physiologic function of the system alone or in combination, thereby adapted to facilitate exudate absorption and hemostasis for internal and external bleeding of the gastrointestinal tract including hemorrhoids and anal fissures.

34. The semisolid system and combination semisolid, multiparticulate system for controlling biological fluids and providing local effects of claim 33, wherein said synergistic compounds include physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, cellulose and cellulose derivatives, hyaluronic and hyaluronic derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes and enzyme inhibitors, said synergistic compounds being operable to facilitate absorption of body fluids, partial or complete clotting of blood, bloody discharge or exudates as an independent semisolid, gel, or liquid mixture.

35. The semisolid system and combination semisolid, multiparticulate system for controlling biological fluids and providing local effects of claim 34, wherein said blood products includes but is not limited to platelets, prothrombin, thrombin, fibrin, fibrinogen, thromboplastin, and all clotting factors.

36. The semisolid system and combination semisolid, multiparticulate system for controlling biological fluids and providing local effects of claim 1, 2, 3, 32, 33 or 34, wherein said semisolid composition is adapted to be incorporated with a standard, commercially available sanitary medical absorbent article.

37. The semisolid system and combination semisolid, multiparticulate system for controlling biological fluids and providing local effects of claim 36, wherein said sanitary medical absorbent article includes but is not limited to a secondary sponge, a wound dressing, a swellable sponge, a fibrotic packing, a wound packing, and a compress, thereby providing an exudate-absorbent, hemostatic tissue sealant article being particularly operable to facilitate exudate absorption and hemostasis for internal and external bleeding of the gastrointestinal tract including hemorrhoids and anal fissures resulting from trauma, hemorrhage, injury, disease or surgical procedures.

38. A semisolid system and combination semisolid, multiparticulate system for controlling biological fluids and providing local effects comprising:
a body fluid moisture equilibrator composition that will form a semisolid line structure in an aqueous or polar solvent environment, said body fluid moisture equilibrator composition being adapted for utilization both as an independent mixture for direct application upon or within body tissues for human or veterinary applications via physical transfer, irrigation, douche, stream, enema or spray and for incorporation with a standard, commercially available medical or catamenial absorbent products in order to provide an optimal moisture balance within, around or upon bodily tissues.

39. The semisolid system and combination semisolid, multiparticulate system for controlling biological fluids and providing local effects of claim 38, wherein said body fluid moisture equilibrator composition comprises a liquid crystal-based system.

40. The semisolid system and combination semisolid, multiparticulate system for controlling biological fluids and providing local effects of claim 38, wherein said body fluid moisture equilibrator composition comprises a greater than 40% fatty acid, fatty acid monoester, fatty acid diester, or fatty acid triester alone or in combination and no less than one of said fatty acid includes at least one unsaturated carbon atom, thereby forming a lipid composition.

41. The semisolid system and combination semisolid, multiparticulate system for controlling biological fluids and providing local effects of claim 40, wherein said semisolid composition comprises 25.0% to 100% by weight of glyceryl monooleate, wherein a balance of said semisolid composition is a solvent, wherein said semisolid composition having a solvent content defined at 0% to 90% polar, 0% to 90% semi-polar, or 0% to 90% nonpolar, thereby providing a mixture being operable to provide an optimal moisture balance within, around or upon bodily tissues by partial absorption and partial evaporation of body fluids as an independent semisolid, gel, or liquid mixture thereby facilitating acceleration of healing rates of the internal and external wounds or burns.

42. The semisolid system and combination semisolid, multiparticulate system for controlling biological fluids and providing local effects of claim 38, wherein said semisolid composition further comprises synergistic compounds or active medicaments used to alter or otherwise strengthen the semisolid structure matrix or consistency, or enhance the physiologic function of the system alone or in combination.

43. The semisolid system and combination semisolid, multiparticulate system for controlling biological fluids and providing local effects of claim 42, wherein said synergistic compounds include physiologically compatible monovalent, divalent and trivalent ions and salts thereof, calcium derivatives, potassium derivatives, sulfate derivatives, phospholipids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, cellulose and cellulose derivatives, hyaluronic and hyaluronic derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes and enzyme inhibitors, said synergistic compounds being operable to facilitate absorption of body fluids, partial or complete clotting of blood, bloody discharge or exudates as an independent semisolid, gel, or liquid mixture.

44. The semisolid system and combination semisolid, multiparticulate system for controlling biological fluids and providing local effects of claim 38, wherein said solvent includes alcohol(s), polyethylene glycol(s), propylene glycol, polypropylene glycol, water, and an isotonic aqueous system or a physiologic buffered aqueous system.

45. The semisolid system and combination semisolid, multiparticulate system for controlling biological fluids and providing local effects of claim 43, wherein said oleaginous ointment base(s) comprises petrolatum, white petrolatum, waxes, cetyl esters wax, oleic acid, olive oil, and spermaceti.

46. The semisolid system and combination semisolid, multiparticulate system for controlling biological fluids and providing local effects of claim 43, wherein said absorbent ointment base(s) comprise lanolin, hydrophilic petrolatum, hydroxystearin sulfate or components thereof.

47. The semisolid system and combination semisolid, multiparticulate system for controlling biological fluids and providing local effects of claim 43, wherein said emulsion ointment base(s) comprise cetyl alcohol, stearic acid, hydrophilic ointment, cold cream or components thereof.

48. The semisolid system and combination semisolid, multiparticulate system for controlling biological fluids and providing local effects of claim 43, wherein said blood products includes but is not limited to platelets, prothrombin, thrombin, fibrin, fibrinogen, thromboplastin, or any clotting factor alone or in combination.

49. The semisolid system and combination semisolid, multiparticulate system for controlling biological fluids and providing local effects of claims 38, 39, 40, 41, 42 or 43, wherein said semisolid composition is adapted to be incorporated with said standard, commercially available medical products, thereby facilitating optimal moisture balance within, around or upon internal and external wounds and burns by partial absorption and partial evaporation of exudate or bodily fluids thereby facilitating acceleration of healing rates of the internal and external wounds or burns.

50. The semisolid system and combination semisolid, multiparticulate system for controlling biological fluids and providing local effects of claim 49, wherein said standard, commercially available medical products includes but is not limited to an occlusive dressing, a non-occlusive dressing, a secondary sponge, a swellable sponge, a fibrotic packing, a wound packing, an adhesive bandage, a disposable diaper or a compress, thereby providing an moisture equilibratory medical article adapted to provide optimal moisture balance within, around or upon internal and external wounds and burns by partial absorption and partial evaporation of bodily fluids.

51. The semisolid system and combination semisolid, multiparticulate system for controlling biological fluids and providing local effects of claim 40, wherein said semisolid composition comprises 25.0% to 100% by weight of glyceryl monooleate, wherein a balance of said semisolid composition is a solvent, wherein said semisolid composition having a solvent content defined at 0% to 90% polar, 0% to 90% semi-polar, or 0% to 90% nonpolar, thereby providing an independent semisolid, gel, or liquid mixture adapted to provide an optimal moisture balance within, around or upon bodily tissues by partial absorption and partial evaporation of bodily fluids, thereby controlling menstrual discharge within, upon, or in close proximity to female genitalia while simultaneously limiting the risk of toxic shock syndrome.

52. The semisolid system and combination semisolid, multiparticulate system for controlling biological fluids and providing local effects of claim 38, wherein said semisolid composition further comprises synergistic compounds or active medicaments used to alter or otherwise strengthen the semisolid structure matrix or consistency, or enhance the physiologic function of the system alone or in combination thereby providing an optimal moisture balance thereby controlling menstrual discharge.

53. The semisolid system and combination semisolid, multiparticulate system for controlling biological fluids and providing local effects of claim 52, wherein said synergistic compounds include physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, cellulose and cellulose derivatives, hyaluronic and hyaluronic derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products including but is not limited to platelets, prothrombin, thrombin, fibrin, fibrinogen, thromboplastin, and all clotting factors; enzymes and enzyme inhibitors, said synergistic compounds being operable to facilitate absorption of body fluids, partial or complete clotting of blood, bloody discharge or exudates as an independent semisolid, gel, or liquid mixture.

54. The semisolid system and combination semisolid, multiparticulate system for controlling biological fluids and providing local effects of claims 38, 39, 40, 51, 52 or 53, wherein said semisolid composition is adapted to be incorporated with a standard, commercially available catamenial absorption article in order to facilitate optimal moisture balance within or upon bodily tissues by partial absorption and partial evaporation of bodily fluids, thereby controlling menstrual discharge within, upon, or in close proximity to female genitalia while simultaneously limiting the risk of toxic shock syndrome.

55. The semisolid system and combination semisolid, multiparticulate system for controlling biological fluids and providing local effects of claim 54, wherein said standard, commercially available catamenial absorption article includes but is not limited to a standard, commercially available feminine tampon, menstrual pad, and feminine napkin, thereby providing an absorbent, equilibratory catamenial article adapted to provide optimal moisture balance within or upon bodily tissues by partial absorption and partial evaporation of bodily fluids, and thereby controlling menstrual discharge within, upon, or in close proximity to female genitalia while simultaneously limiting the risk of toxic shock syndrome.

56. The semisolid system and combination semisolid, multiparticulate system for controlling biological fluids and providing local effects of claim 40, wherein said semisolid composition comprises 25.0% to 100% by weight of glyceryl monooleate, wherein a balance of said semisolid composition is a solvent, wherein said semisolid composition having a solvent content defined at 0% to 90% polar, 0% to 90% semi-polar, or 0% to 90% nonpolar, thereby providing an independent semisolid, gel, or liquid mixture adapted to provide an optimal moisture balance within, around or upon bodily tissues by partial absorption and partial evaporation of bodily fluids, thereby improving cosmetic outcomes of normal and hypertrophic scars resulting from trauma, disease, or surgical procedures.

57. The semisolid system and combination semisolid, multiparticulate system for controlling biological fluids and providing local effects of claim 38, wherein said semisolid composition further comprises synergistic compounds or active medicaments used to alter or otherwise strengthen the semisolid structure matrix or consistency, or enhance the physiologic function of the system alone or in combination, thereby adapted to provide an optimal moisture balance thereby improving cosmetic outcomes of normal and hypertrophic scars.

58. The semisolid system and combination semisolid, multiparticulate system for controlling biological fluids and providing local effects of claim 57, wherein said synergistic compounds include onion extracts, fatty acids, silicone derivative(s), astringent agent(s), UV radiation blocker(s) and vasoconstrictors, and wherein said active medicaments include anti-inflammatory agents, including steroids and non-steroidal anti-inflammatory drugs and proteins.

59. The semisolid system and combination semisolid, multiparticulate system for controlling biological fluids and providing local effects of claims 38, 39, 40, 56, 57, or 58, wherein said semisolid composition is adapted to be incorporated with a standard, commercially available medical product, thereby facilitating optimal moisture balance within, around or upon bodily tissues by partial absorption and partial evaporation of bodily fluids, thereby improving cosmetic outcomes for normal and hypertrophic scars resulting from trauma, disease, or surgical procedures.

60. The semisolid system and combination semisolid, multiparticulate system for controlling biological fluids and providing local effects of claim 59, wherein said standard, commercially available medical product includes but is not limited to a secondary sponge, a wound dressing, a swellable sponge, a fibrotic packing, a wound packing, an adhesive bandage, an adhesive bandage, and a compress, thereby providing a moisture equilibratory article adapted to provide optimal moisture balance within, around or upon bodily tissues by partial absorption and partial evaporation of bodily fluids, thereby improving cosmetic outcomes for normal and hypertrophic scars resulting from trauma, disease, or surgical procedures.

61. The semisolid system and combination semisolid, multiparticulate system for controlling biological fluids and providing local effects of claim 40, wherein said semisolid composition comprises 25.0% to 100% by weight of glyceryl monooleate, wherein a balance of said semisolid composition is a solvent, wherein said semisolid composition having a solvent content defined at 0% to 90% polar, 0% to 90% semi-polar, or 0% to 90% nonpolar, thereby providing a mixture being operable to provide an optimal moisture balance within or upon bodily tissues by partial absorption and partial evaporation of body fluids as an independent semisolid, gel, or liquid mixture, in order to further provide lubrication and treatment of hemorrhoids and anal fissures before and after surgical intervention.

62. The semisolid system and combination semisolid, multiparticulate system for controlling biological fluids and providing local effects of claim 38, wherein said semisolid composition further comprises synergistic compounds or active medicaments used to alter or otherwise strengthen the semisolid structure matrix or consistency, or enhance the physiologic function of the system alone or in combination, thereby providing an optimal moisture balance in order to further provide lubrication and treatment of hemorrhoids and anal fissures.

63. The semisolid system and combination semisolid, multiparticulate system for controlling biological fluids and providing local effects of claim 62, wherein said synergistic compounds include physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, cellulose and cellulose derivatives, hyaluronic and hyaluronic derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products including but is not limited to platelets, prothrombin, thrombin, fibrin, fibrinogen, thromboplastin, and all clotting factors; enzymes and enzyme inhibitors, said synergistic compounds being operable to facilitate absorption of body fluids, partial or complete clotting of blood, bloody discharge or exudates as an independent semisolid, gel, or liquid mixture.

64. The semisolid system and combination semisolid, multiparticulate system for controlling biological fluids and providing local effects of claim 38, 39, 40, 61, 62, or 63, wherein said semisolid composition is adapted to be incorporated with a standard, commercially available medical product, thereby facilitating optimal moisture balance within, around or upon bodily tissues by partial absorption and partial evaporation of body fluids and providing lubrication and treatment of hemorrhoids and anal fissures before and after surgical intervention.

65. The semisolid system and combination semisolid, multiparticulate system for controlling biological fluids and providing local effects of claim 64, wherein said standard, commercially available medical product includes but is not limited to a secondary sponge, a wound dressing, a swellable sponge, a fibrotic packing, a wound packing, and a compress, thereby providing an absorbent, anti-infective, lubricant product adapted to provide optimal moisture balance within, around or upon bodily tissues.

66. The semisolid system and combination semisolid, multiparticulate system for controlling biological fluids and providing local effects of claim 40, wherein said semisolid composition comprises 25.0% to 100% by weight of glyceryl monooleate, wherein a balance of said semisolid composition is a solvent, wherein said semisolid composition having a solvent content defined at 0% to 90% polar, 0% to 90% semi-polar, or 0% to 90% nonpolar, thereby providing a mixture being operable to provide an optimal moisture balance within or upon bodily tissues within or near the oropharangeal region including the mouth, teeth, gums or throat by partial absorption and partial evaporation of body fluids as an independent semisolid, gel, or liquid mixture, operable in periodontal treatment or procedures.

67. The semisolid system and combination semisolid, multiparticulate system for controlling biological fluids and providing local effects of claim 38, wherein said semisolid composition further comprises synergistic compounds or active medicaments used to alter or otherwise strengthen the semisolid structure matrix or consistency, or enhance the physiologic function of the system alone or in combination, thereby providing an optimal moisture balance within or upon bodily tissues within or near the oropharangeal region.

68. The semisolid system and combination semisolid, multiparticulate system for controlling biological fluids and providing local effects of claim 67, wherein said synergistic compounds include physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, teeth whitening agents, cellulose and cellulose derivatives, hyaluronic and hyaluronic derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products including but is not limited to platelets, prothrombin, thrombin, fibrin, fibrinogen, thromboplastin, and all clotting factors; enzymes and enzyme inhibitors, said synergistic compounds being operable to facilitate absorption of body fluids, partial or complete clotting of blood, bloody discharge or exudates as an independent semisolid, gel, or liquid mixture.

69. The semisolid system and combination semisolid, multiparticulate system for controlling biological fluids and providing local effects of claims 38, 39, 40, 66, 67 or 68, wherein said semisolid composition is adapted to be incorporated with a standard, commercially available medical product or dental mouth piece or tray, in order to provide optimal moisture balance with dental or periodontal treatment procedures or as a component of a dental kit.

70. The semisolid system and combination semisolid, multiparticulate system for controlling biological fluids and providing local effects of claim 69, wherein said standard, commercially available medical product includes but is not limited to a secondary sponge, a wound dressing, a swellable sponge, a fibrotic packing, a wound packing, and a compress.

71. A semisolid system and combination semisolid, multiparticulate system for controlling biological fluids and providing local effects comprising:
an anti-infective composition that will form a semisolid line structure in an aqueous or polar solvent environment, said anti-infective composition being adapted for utilization both as an independent mixture for direct application upon or within body tissues for human or veterinary applications via physical transfer, irrigation, douche, stream or spray and for incorporation with standard, commercially available medical or surgical articles being operable as an anti-infective agent, thereby inhibiting the production of and serving to treat or prevent infections caused by bacteria, viruses, protozoa, fungi, yeast, and analogous causative agents of infectious diseases.

72. The semisolid system and combination semisolid, multiparticulate system for controlling biological fluids and providing local effects of claim 71, wherein said anti-infective composition comprises a liquid crystal-based system.

73. The semisolid system and combination semisolid, multiparticulate system for controlling biological fluids and providing local effects of claim 71, wherein said anti-infective composition comprises a greater than 40% fatty acid, fatty acid monoester, fatty acid diester, or fatty acid triester alone or in combination and no less than one of said fatty acid includes at least one unsaturated carbon atom, thereby forming a lipid composition.

74. The semisolid system and combination semisolid, multiparticulate system for controlling biological fluids and providing local effects of claim 73, wherein said semisolid composition comprises 25.0% to 100% by weight of glyceryl monooleate, wherein a balance of said semisolid composition is a solvent, wherein said semisolid composition having a solvent content defined at 0% to 90% polar, 0% to 90% semi-polar, or 0% to 90% nonpolar, thereby providing a mixture being operable to function as an anti-infective agent within or upon bodily tissues as an independent semisolid, gel, or liquid mixture.

75. The semisolid system and combination semisolid, multiparticulate system for controlling biological fluids and providing local effects of claim 71, wherein said semisolid composition further comprises synergistic compounds or active medicaments used to alter or otherwise strengthen the semisolid structure matrix or consistency, or enhance the physiologic function of the system alone or in combination.

76. The semisolid system and combination semisolid, multiparticulate system for controlling biological fluids and providing local effects of claim 75, wherein said synergistic compounds include physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, cellulose and cellulose derivatives, hyaluronic and hyaluronic derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes and enzyme inhibitors, said synergistic compounds being operable to facilitate absorption of body fluids, partial or complete clotting of blood, bloody discharge or exudates as an independent semisolid, gel, or liquid mixture.

77. The semisolid system and combination semisolid, multiparticulate system for controlling biological fluids and providing local effects of claim 71, wherein said solvent includes alcohol(s), polyethylene glycol(s), propylene glycol, polypropylene glycol, water, and an isotonic aqueous system or a physiologic buffered aqueous system.

78. The semisolid system and combination semisolid, multiparticulate system for controlling biological fluids and providing local effects of claim 76, wherein said oleaginous ointment base(s) comprises petrolatum, white petrolatum, waxes, cetyl esters wax, oleic acid, olive oil, and spermaceti as nonlimiting examples.

79. The semisolid system and combination semisolid, multiparticulate system for controlling biological fluids and providing local effects of claim 76, wherein said absorbent ointment base(s) comprise lanolin, hydrophilic petrolatum, hydroxystearin sulfate or components thereof as nonlimiting examples.

80. The semisolid system and combination semisolid, multiparticulate system for controlling biological fluids and providing local effects of claim 76, wherein said emulsion ointment base(s) comprise cetyl alcohol, stearic acid, hydrophilic ointment, cold cream or components thereof as nonlimiting examples.

81. The semisolid system and combination semisolid, multiparticulate system for controlling biological fluids and providing local effects of claim 76, wherein said blood products includes but is not limited to platelets, prothrombin, thrombin, fibrin, fibrinogen, thromboplastin, and all clotting factors.

82. The semisolid system and combination semisolid, multiparticulate system for controlling biological fluids and providing local effects of claims 71, 72, 73, 74, 75, or 76, wherein said semisolid composition is adapted to be incorporated with a standard, commercially available medical product in order to provide an anti-infective effect for internal and external wounds or burns resulting from trauma, hemorrhage, injury, disease, or surgical procedures, thereby inhibiting the production of and serving to treat or prevent infections caused by bacteria, viruses, protozoa, fungi, yeast, and analogous causative agents of infectious diseases.

83. The semisolid system and combination semisolid, multiparticulate system for controlling biological fluids and providing local effects of claim 82, wherein said standard, commercially available medical product includes but is not limited to a secondary sponge, a wound dressing, a swellable sponge, a fibrotic packing, a wound packing, an adhesive bandage, a compress, an implantable medical device, implantable bio-hardware and implantable prosthetic hardware, thereby providing an anti-infective effect for internal and external wounds or burns resulting from trauma, hemorrhage, injury, disease, or surgical procedures, thereby inhibiting the production of and serving to treat infection caused by bacteria, viruses, protozoa, fungi, yeast, and analogous causative agents of infectious diseases.

84. The semisolid system and combination semisolid, multiparticulate system for controlling biological fluids and providing local effects of claim 73, wherein said semisolid composition comprises 25.0% to 100% by weight of glyceryl monooleate, wherein a balance of said semisolid composition is a solvent, wherein said semisolid composition having a solvent content defined at 0% to 90% polar, 0% to 90% semi-polar, or 0% to 90% nonpolar, thereby providing a mixture being operable for use in or upon the gastrointestinal tract including hemorrhoids and anal fissures prior to and following surgical procedures through the anti-infective effect as an independent semisolid, gel, or liquid mixture.

85. The semisolid system and combination semisolid, multiparticulate system for controlling biological fluids and providing local effects of claim 71, wherein said semisolid composition further comprises synergistic compounds or active medicaments used to alter or otherwise strengthen the semisolid structure matrix or consistency, or enhance the physiologic function of the system alone or in combination, thereby providing an anti-infective mixture being operable for use in or upon the gastrointestinal tract including hemorrhoids and anal fissures.

86. The semisolid system and combination semisolid, multiparticulate system for controlling biological fluids and providing local effects of claim 85, wherein said synergistic compounds include physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, cellulose and cellulose derivatives, hyaluronic and hyaluronic derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products including but is not limited to platelets, prothrombin, thrombin, fibrin, fibrinogen, thromboplastin, and all clotting factors; enzymes and enzyme inhibitors, said synergistic compounds being operable to facilitate absorption of body fluids, partial or complete clotting of blood, bloody discharge or exudates as an independent semisolid, gel, or liquid mixture.

87. The semisolid system and combination semisolid, multiparticulate system for controlling biological fluids and providing local effects of claims 71, 72, 73, 84, 85, or 86, wherein said semisolid composition is adapted to be incorporated with a standard, commercially available medical product being operable for use in or upon the gastrointestinal tract including hemorrhoids and anal fissures prior to or following treatment or surgical procedures through the anti-infective effect.

88. The semisolid system and combination semisolid, multiparticulate system for controlling biological fluids and providing local effects of claim 87, wherein said standard, commercially available medical product includes but is not limited to a secondary sponge, a wound dressing, a swellable sponge, a fibrotic packing, a wound packing, and a compress, thereby providing an anti-infective product adapted for use in or upon the gastrointestinal tract including hemorrhoids and anal fissures prior to and following treatment or surgical procedures through the anti-infective effect.

89. The semisolid system and combination semisolid, multiparticulate system for controlling biological fluids and providing local effects of claim 73, wherein said semisolid composition comprises 25.0% to 100% by weight of glyceryl monooleate, wherein a balance of said semisolid composition is a solvent, wherein said semisolid composition having a solvent content defined at 0% to 90% polar, 0% to 90% semi-polar, or 0% to 90% nonpolar, thereby providing a mixture being operable with dental or periodontal treatment or procedures resulting from trauma, hemorrhage, injury, or disease through the anti-infective effect as an independent semisolid, gel, or liquid mixture.

90. The semisolid system and combination semisolid, multiparticulate system for controlling biological fluids and providing local effects of claim 71, wherein said semisolid composition further comprises synergistic compounds or active medicaments used to alter or otherwise strengthen the semisolid structure matrix or consistency, or enhance the physiologic function of the system alone or in combination, thereby providing a mixture being operable with dental or periodontal treatment or procedures.

91. The semisolid system and combination semisolid, multiparticulate system for controlling biological fluids and providing local effects of claim 90, wherein said synergistic compounds include physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, cellulose and cellulose derivatives, hyaluronic and hyaluronic derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products including but is not limited to platelets, prothrombin, thrombin, fibrin, fibrinogen, thromboplastin, and all clotting factors, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products; enzymes and enzyme inhibitors, said synergistic compounds being operable to facilitate absorption of body fluids, partial or complete clotting of blood, bloody discharge or exudates as an independent semisolid, gel, or liquid mixture.

92. The semisolid system and combination semisolid, multiparticulate system for controlling biological fluids and providing local effects of claims 71, 72, 73, 89, 90, or 91, wherein said semisolid composition is adapted to be incorporated with a standard, commercially available medical product or dental mouthpiece or tray, in order to facilitate dental or periodontal treatment or procedures resulting from trauma, hemorrhage, injury, or disease through the anti-infective effect or as a component of a dental kit.

93. The semisolid system and combination semisolid, multiparticulate system for controlling biological fluids and providing local effects of claim 92, wherein said standard, commercially available medical product includes but is not limited to a secondary sponge, a wound dressing, a swellable sponge, a fibrotic packing, a wound packing, and a compress.

94. The semisolid system and combination semisolid, multiparticulate system for controlling biological fluids and providing local effects of claim 73, wherein said semisolid composition of comprises 25.0% to 100% by weight of glyceryl monooleate, wherein a balance of said semisolid composition is a solvent, wherein said semisolid composition having a solvent content defined at 0% to 90% polar, 0% to 90% semi-polar, or 0% to 90% nonpolar, said composition being adapted to function an anti-infective coating upon, but not limited to, implantable medical devices, implantable bio-hardware and implantable prosthetic hardware and wherein said semisolid composition being further adapted for use as an independent semisolid, gel, or liquid mixture.

95. The semisolid system and combination semisolid, multiparticulate system for controlling biological fluids and providing local effects of claim 71, wherein said semisolid composition further comprises synergistic compounds or active medicaments used to alter or otherwise strengthen the semisolid structure matrix or consistency, or enhance the physiologic function of the system alone or in combination, thereby adapted to function an anti-infective coating.

96. The semisolid system and combination semisolid, multiparticulate system for controlling biological fluids and providing local effects of claim 95, wherein said synergistic compounds include physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, cellulose and cellulose derivatives, hyaluronic and hyaluronic derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products including but is not limited to platelets, prothrombin, thrombin, fibrin, fibrinogen, thromboplastin, and all clotting factors; enzymes, enzyme inhibitors, and periodontal agents said synergistic compounds being operable to facilitate absorption of body fluids, partial or complete clotting of blood, bloody discharge or exudates as an independent semisolid, gel, or liquid mixture.

97. The semisolid system and combination semisolid, multiparticulate system for controlling biological fluids and providing local effects of claims 71, 72, 73, 94, 95, or 96, wherein said semisolid composition is adapted to be incorporated with standard, commercially available orthopedic or dental hardware, wherein said orthopedic or dental hardware being specifically adapted for implantation into the body.

98. The semisolid system and combination semisolid, multiparticulate system for controlling biological fluids and providing local effects of claim 97, wherein said orthopedic or dental hardware includes but is not limited to joint or partial limb prosthetics, dental prosthetics and devices, stents, shunts, and related dental, medical and orthopedic hardware.

99. The semisolid system and combination semisolid, multiparticulate system for controlling biological fluids and providing local effects of claim 73, wherein said semisolid composition comprises 25.0% to 100% by weight of glyceryl monooleate, wherein a balance of said semisolid composition is a solvent, wherein said semisolid composition having a solvent content defined at 0% to 90% polar, 0% to 90% semi-polar, or 0% to 90% nonpolar, said semisolid composition functions to provide a sexually transmitted disease prevention lubricant adapted for use as an independent semisolid, gel, or liquid mixture.

100. The semisolid system and combination semisolid, multiparticulate system for controlling biological fluids and providing local effects of claim 71, wherein said semisolid composition further comprises synergistic compounds or active medicaments used to alter or otherwise strengthen the semisolid structure matrix or consistency, or enhance the physiologic function of the system alone or in combination, thereby providing a sexually transmitted disease prevention lubricant.

101. The semisolid system and combination semisolid, multiparticulate system for controlling biological fluids and providing local effects of claim 100, wherein said synergistic compounds include physiologically compatible monovalent, divalent and trivalent ions and salts thereof, calcium derivatives, potassium derivatives, sulfate derivatives, phospholipids, fatty acids, bentonite, fumed silica, colloidal silica, micronized silica, diatomaceous earth, talc, titanium dioxide, potassium aluminum sulfate, aluminum chloride, ammonium chloride ferric sulfate, ferric sub sulfate, copper sulfate, gelatin and gelatin derivatives, cellulose and cellulose derivatives, hyaluronic and hyaluronic derivatives, collagen, chitosan derivatives, hydrogel forming agents, hydrocolloid forming agents, alginic acid derivatives, oleaginous ointment bases, absorbent ointment bases, emulsion ointment bases, astringent agents, vasoconstrictors, proteins, and blood products, and wherein said active medicaments include anti-infective agents including antibiotics, antifungals, and antivirals; anti-inflammatory agents including steroids and non-steroidal anti-inflammatory drugs; sympathomimetic agents; anesthetic agents; analgesic agents; blood products including but is not limited to platelets, prothrombin, thrombin, fibrin, fibrinogen, thromboplastin, and all clotting factors; enzymes, enzyme inhibitors, gingivitis agents, periodontal agents, and fluoride derivatives with said synergistic compounds being operable to facilitate absorption of body fluids, partial or complete clotting of blood, bloody discharge or exudates as an independent semisolid, gel, or liquid mixture.

102. The semisolid system and combination semisolid, multiparticulate system for controlling biological fluids and providing local effects of claims 71, 72, 73, 99, 100, or 101, wherein said semisolid composition is adapted to be incorporated with standard, commercially available birth control or prophylactic devices.

103. The semisolid system and combination semisolid, multiparticulate system for controlling biological fluids and providing local effects of claim 102, wherein said standard, commercially available birth control or prophylactic devices include but are not limited to condoms, diaphragms, and related contraceptive barrier devices and products.

104. The semisolid system and combination semisolid, multiparticulate system for controlling biological fluids and providing local effects of claim 1, wherein said semisolid system and combination semisolid, multiparticulate system being adapted for utilization as an inherent Thrombin inhibitor, operable as an adjunct in the treatment of malignant tumors.

105. The semisolid system and combination semisolid, multiparticulate system for controlling biological fluids and providing local effects of claim 3, wherein said semisolid system and combination semisolid, multiparticulate system being adapted for utilization as an inherent Thrombin inhibitor, operable as an adjunct in the treatment of malignant tumors.

106. The semisolid system and combination semisolid, multiparticulate system for controlling biological fluids and providing local effects of claim 1, wherein said semisolid system and combination semisolid, multiparticulate system being adapted for utilization as an inherent neuroprotective agent, operable to reduce thrombin related inflammation and cellular death of neural tissue.

107. The semisolid system and combination semisolid, multiparticulate system for controlling biological fluids and providing local effects of claim 3, wherein said semisolid system and combination semisolid, multiparticulate system being adapted for utilization as an inherent neuroprotective agent, operable to reduce thrombin related inflammation and cellular death of neural tissue.

108. The semisolid system and combination semisolid, multiparticulate system for controlling biological fluids and providing local effects of claim 1, wherein said semisolid system and combination semisolid, multiparticulate system being adapted for utilization as a vascular plug, operable to halt blood and exudate flow.

109. The semisolid system and combination semisolid, multiparticulate system for controlling biological fluids and providing local effects of claim 3, wherein said semisolid system and combination semisolid, multiparticulate system being adapted for utilization as a vascular plug, operable to halt blood and exudate flow.

110. The semisolid system and combination semisolid, multiparticulate system for controlling biological fluids and providing local effects of claim 1, wherein said semisolid system and combination semisolid, multiparticulate system being adapted for utilization as a biocompatible injection or fill media for cosmetic and functional surgical augmentations including but not limited to lip injections, wrinkle injections, synovial injections and breast implant fill media.

111. The semisolid system and combination semisolid, multiparticulate system for controlling biological fluids and providing local effects of claim 3, wherein said semisolid system and combination semisolid, multiparticulate system being adapted for utilization as a biocompatible injection or fill media for cosmetic and functional surgical augmentations including but not limited to lip injections, wrinkle injections, synovial injections and breast implant fill media.

112. The semisolid system and combination semisolid, multiparticulate system for controlling biological fluids and providing local effects of claim 1, further comprised of a mechanism of enhanced hemostasis facilitated by alteration of the local microenvironment at a site of hemorrhage resulting in a direct or indirect enhancement of an action of constituents of an intrinsic pathway in a coagulation cascade.

113. The semisolid system and combination semisolid, multiparticulate system for controlling biological fluids and providing local effects of claim 3, further comprised of a mechanism of enhanced hemostasis facilitated by alteration of the local microenvironment at a site of hemorrhage resulting in a direct or indirect enhancement of an action of constituents of an intrinsic pathway in a coagulation cascade.

114. The semisolid system and combination semisolid, multiparticulate system for controlling biological fluids and providing local effects of claim 1, further comprised of a mechanism of enhanced hemostasis facilitated by alteration of the local microenvironment at a site of hemorrhage resulting in a direct or indirect enhancement of an action of constituents of an extrinsic pathway in a coagulation cascade.

115. The semisolid system and combination semisolid, multiparticulate system for controlling biological fluids and providing local effects of claim 3, further comprised of a mechanism of enhanced hemostasis facilitated by alteration of the local microenvironment at a site of hemorrhage resulting in a direct or indirect enhancement of an action of constituents of an extrinsic pathway in a coagulation cascade.

116. The semisolid system and combination semisolid, multiparticulate system for controlling biological fluids and providing local effects of claim 1, further comprised of a mechanism of enhanced hemostasis facilitated by alteration of the local microenvironment at a site of hemorrhage resulting in a direct or indirect enhancement of an action of constituents of an common pathway in a coagulation cascade.

117. The semisolid system and combination semisolid, multiparticulate system for controlling biological fluids and providing local effects of claim 3, further comprised of a mechanism of enhanced hemostasis facilitated by alteration of the local microenvironment at a site of hemorrhage resulting in a direct or indirect enhancement of an action of constituents of an common pathway in a coagulation cascade.

118. The semisolid system and combination semisolid, multiparticulate system for controlling biological fluids and providing local effects of claim 1, further comprised of a mechanism of enhanced hemostasis facilitated by alteration of the local microenvironment at a site of hemorrhage resulting in a direct or indirect concentration of platelets.

119. The semisolid system and combination semisolid, multiparticulate system for controlling biological fluids and providing local effects of claim 3, further comprised of a mechanism of enhanced hemostasis facilitated by alteration of the local microenvironment at a site of hemorrhage resulting in a direct or indirect concentration of platelets.

120. The semisolid system and combination semisolid, multiparticulate system for controlling biological fluids and providing local effects of claim 1, further comprised of a mechanism of enhanced hemostasis facilitated by alteration of the local microenvironment at a site of hemorrhage resulting in a direct or indirect concentration of activation proteins.

121. The semisolid system and combination semisolid, multiparticulate system for controlling biological fluids and providing local effects of claim 3, further comprised of a mechanism of enhanced hemostasis facilitated by alteration of the local microenvironment at a site of hemorrhage resulting in a direct or indirect activation of proteins or by concentration of activation proteins.

122. A semisolid system and combination semisolid, multiparticulate system for delivering active compounds and medicaments to target tissues comprising:
a body fluid control composition that will form or maintain a semisolid secondary structure in an aqueous or polar solvent environment, said body fluid control composition being adapted for utilization both as an independent mixture for direct application upon or within body tissues for human or veterinary applications via direct application, trans-dermal delivery, injected deposit, oral delivery or implantation in order to facilitate targeted delivery of the active compound or medicament to the target location or tissue.

123. The semisolid system and combination semisolid, multiparticulate system delivering active compounds and medicaments to target tissues of claim 122, wherein said active compounds and medicaments include, but are not limited to, stem cells, immunogenic agents, biogenic factors and hormones including, but not limited to, hormones utilized for birth control.

124. The semisolid system and combination semisolid, multiparticulate system for controlling biological fluids and providing local effects of claim 3, wherein said semisolid is a fatty acid containing 8 to 20 carbons, a monoglyceride containing 8 to 25 carbons or a diglyceride containing 10 to 45 carbons.

125. The semisolid system and combination semisolid, multiparticulate system for controlling biological fluids and providing local effects of claim 40 wherein said semisolid is a fatty acid containing 8 to 20 carbons, a monoglyceride containing 8 to 25 carbons or a diglyceride containing 10 to 45 carbons.

126. The semisolid system and combination semisolid, multiparticulate system for controlling biological fluids and providing local effects of claim 73 wherein said semisolid is a fatty acid containing 8 to 20 carbons, a monoglyceride containing 8 to 25 carbons or a diglyceride containing 10 to 45 carbons.

127. The semisolid system and combination semisolid, multiparticulate system for controlling biological fluids and providing local effects of claim 3, wherein said semisolid composition is utilized at least partially or exclusively for its inherent lubricating or anti-adherent features for a medical absorbent article.

128. A method of manufacturing a semisolid system and combination semisolid, multiparticulate system, comprising the steps of:
(1) utilizing microwave radiation to assist heterogeneous mixing and processing of a composition to assist conversion thereof to a desired consistency, wherein said composition includes a semisolid composition and other various components adapted for mixture with a selected said composition.
